SEQUENCE LISTING (23 sequences)

SEQ ID NO: 1 - DNA, 3187 bp, synthetic construct
  misc_feature 1..3187: note = vector genome AAV.CMVe.chTNTp.LaminA.RBG
  repeat_region 1..130: note = 5' ITR
  regulatory 191..572: note = enhancer - CMV IE enhancer; regulatory_class = enhancer
  regulatory 582..808: note = promoter - TnT promoter; regulatory_class = promoter
  misc_feature 827..2771: note = LaminA
  regulatory 2843..2969: note = polyA_signal - Rabbit globin polyA; regulatory_class = polyA_signal_sequence
  repeat_region 3058..3187: note = 3' ITR
  source 1..3187: mol_type = other DNA
  misc_feature 191..808: note = hybrid CMV IE enhancer with chicken cardiac troponin T promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagactagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaatagcttgggcttatatgggctcctgtgggggaagggggagcacggagggggccggggccgctgctgccaaaatagcagctcacaagtgttgcattcctctctgggcgccgggcacattcctgctgctctgcccgccccggggtgggcgccggggggaccttaaagcctctgccccccaaggagcccttcccagatagccgccggcacccaccgctccgtgggacctcggcacaagatctgaattcgccaccatggaaacccctagccagcggagagctacaagaagcggagcccaggcctctagcacacctctgagccctaccagaatcacccggctgcaagagaaagaggacctgcaagagctgaacgaccggctggccgtgtacatcgacagagtcagaagcctggaaaccgagaacgccggcctgagactgagaatcaccgagagcgaggaagtggtgtccagagaggtgtccggaatcaaggccgcctacgaagccgaactgggcgacgccagaaagaccctggatagcgtggccaaagagcgggccagactgcagctggaactgagcaaagtgcgggaagagttcaaagaactgaaggcccggaacaccaagaaagagggcgacctgattgccgctcaggccaggctgaaggatctggaagctctgctgaacagcaaagaggccgctctgagcacagccctgagcgagaagagaacactggaaggcgagctgcacgacctgagaggacaggtggcaaaactggaagccgctctgggagaagccaagaagcagctgcaggacgagatgctgagaagagtggacgccgagaaccggctgcagaccatgaaggaagaactggacttccagaagaacatctacagcgaggaactgcgcgagacaaagcggcggcacgagacaagactggtggaaatcgacaacggcaagcagagagagttcgagagcagactggccgacgctctgcaagaactgagagcccagcacgaggaccaggtggaacagtataagaaagagctggaaaagacctacagcgccaagctggacaacgccagacagagcgccgagagaaacagcaatcttgtgggagccgctcacgaagaactgcagcagagccggatcagaatcgacagcctgtctgcccagctgtctcagctgcagaaacagctggccgccaaagaggctaagctgcgggacctcgaagattccctggccagagagagagacaccagcagaaggctgctggccgagaaagaacgcgagatggccgagatgagagcccggatgcagcagcagctggacgagtatcaagagctgctggacatcaagctggccctggacatggaaatccacgcctaccggaaactgctcgaaggcgaggaagagagactgcggctgagcccatctcctaccagccagagaagtagaggcagagcctccagccacagcagccaaacacaaggcggaggcagcgtgaccaagaagaggaagctggaaagcaccgagtccagatccagcttcagccagcacgccagaacatctggcagagtggccgtggaagaggtggacgaagagggcaaattcgtgcggctgcggaacaagagcaacgaggatcagagcatgggcaactggcagatcaagcggcagaacggcgacgatcccctgctgacctaccggttccctccaaagttcaccctgaaggccggccaggtggtcacaatttgggcagctggtgctggcgccacacactctccacctactgatctcgtgtggaaggcccagaatacctggggctgtggcaacagcctgagaactgccctgatcaacagcaccggcgaagaggtcgccatgagaaaacttgtgcggagcgtgaccgtggtggaagacgacgaggatgaggatggcgacgacctgctgcaccaccaccacggatctcactgtagcagctctggcgatcccgccgagtacaacctgagaagcagaaccgtgctgtgcggcacatgtggacagcctgccgataaggcctctgctagtggatctggcgcccaagttggcggccctatcagctctggatctagcgcctctagcgtgacagtgacccggtcctatagaagcgttggaggaagcggaggcggcagcttcggcgataatctggtcaccagaagctactgatgactcgagactagtcaattggtcgacctcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 2779 bp, synthetic construct
  misc_feature 1..2779: note = expression cassette CMVe.chTNTp.LaminA.RBG
  regulatory 1..382: note = enhancer - CMV IE enhancer; regulatory_class = enhancer
  regulatory 392..618: note = promoter - TnT promoter; regulatory_class = promoter
  regulatory 2653..2779: note = polyA_signal - Rabbit globin polyA; regulatory_class = polyA_signal_sequence
  source 1..2779: mol_type = other DNA
  CDS 637..2577: note = Lamin A; protein_id = 3; translation = METPSQRRATRSGAQASSTPLSPTRITRLQEKEDLQELNDRLAV YIDRVRSLETENAGLRLRITESEEVVSREVSGIKAAYEAELGDARKTLDSVAKERARL QLELSKVREEFKELKARNTKKEGDLIAAQARLKDLEALLNSKEAALSTALSEKRTLEG ELHDLRGQVAKLEAALGEAKKQLQDEMLRRVDAENRLQTMKEELDFQKNIYSEELRET KRRHETRLVEIDNGKQREFESRLADALQELRAQHEDQVEQYKKELEKTYSAKLDNARQ SAERNSNLVGAAHEELQQSRIRIDSLSAQLSQLQKQLAAKEAKLRDLEDSLARERDTS RRLLAEKEREMAEMRARMQQQLDEYQELLDIKLALDMEIHAYRKLLEGEEERLRLSPS PTSQRSRGRASSHSSQTQGGGSVTKKRKLESTESRSSFSQHARTSGRVAVEEVDEEGK FVRLRNKSNEDQSMGNWQIKRQNGDDPLLTYRFPPKFTLKAGQVVTIWAAGAGATHSP PTDLVWKAQNTWGCGNSLRTALINSTGEEVAMRKLVRSVTVVEDDEDEDGDDLLHHHH GSHCSSSGDPAEYNLRSRTVLCGTCGQPADKASASGSGAQVGGPISSGSSASSVTVTR SYRSVGGSGGGSFGDNLVTRSY
  misc_feature 1..618: note = hybrid CMV IE enhancer with chicken cardiac troponin T promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaatagcttgggcttatatgggctcctgtgggggaagggggagcacggagggggccggggccgctgctgccaaaatagcagctcacaagtgttgcattcctctctgggcgccgggcacattcctgctgctctgcccgccccggggtgggcgccggggggaccttaaagcctctgccccccaaggagcccttcccagatagccgccggcacccaccgctccgtgggacctcggcacaagatctgaattcgccaccatggaaacccctagccagcggagagctacaagaagcggagcccaggcctctagcacacctctgagccctaccagaatcacccggctgcaagagaaagaggacctgcaagagctgaacgaccggctggccgtgtacatcgacagagtcagaagcctggaaaccgagaacgccggcctgagactgagaatcaccgagagcgaggaagtggtgtccagagaggtgtccggaatcaaggccgcctacgaagccgaactgggcgacgccagaaagaccctggatagcgtggccaaagagcgggccagactgcagctggaactgagcaaagtgcgggaagagttcaaagaactgaaggcccggaacaccaagaaagagggcgacctgattgccgctcaggccaggctgaaggatctggaagctctgctgaacagcaaagaggccgctctgagcacagccctgagcgagaagagaacactggaaggcgagctgcacgacctgagaggacaggtggcaaaactggaagccgctctgggagaagccaagaagcagctgcaggacgagatgctgagaagagtggacgccgagaaccggctgcagaccatgaaggaagaactggacttccagaagaacatctacagcgaggaactgcgcgagacaaagcggcggcacgagacaagactggtggaaatcgacaacggcaagcagagagagttcgagagcagactggccgacgctctgcaagaactgagagcccagcacgaggaccaggtggaacagtataagaaagagctggaaaagacctacagcgccaagctggacaacgccagacagagcgccgagagaaacagcaatcttgtgggagccgctcacgaagaactgcagcagagccggatcagaatcgacagcctgtctgcccagctgtctcagctgcagaaacagctggccgccaaagaggctaagctgcgggacctcgaagattccctggccagagagagagacaccagcagaaggctgctggccgagaaagaacgcgagatggccgagatgagagcccggatgcagcagcagctggacgagtatcaagagctgctggacatcaagctggccctggacatggaaatccacgcctaccggaaactgctcgaaggcgaggaagagagactgcggctgagcccatctcctaccagccagagaagtagaggcagagcctccagccacagcagccaaacacaaggcggaggcagcgtgaccaagaagaggaagctggaaagcaccgagtccagatccagcttcagccagcacgccagaacatctggcagagtggccgtggaagaggtggacgaagagggcaaattcgtgcggctgcggaacaagagcaacgaggatcagagcatgggcaactggcagatcaagcggcagaacggcgacgatcccctgctgacctaccggttccctccaaagttcaccctgaaggccggccaggtggtcacaatttgggcagctggtgctggcgccacacactctccacctactgatctcgtgtggaaggcccagaatacctggggctgtggcaacagcctgagaactgccctgatcaacagcaccggcgaagaggtcgccatgagaaaacttgtgcggagcgtgaccgtggtggaagacgacgaggatgaggatggcgacgacctgctgcaccaccaccacggatctcactgtagcagctctggcgatcccgccgagtacaacctgagaagcagaaccgtgctgtgcggcacatgtggacagcctgccgataaggcctctgctagtggatctggcgcccaagttggcggccctatcagctctggatctagcgcctctagcgtgacagtgacccggtcctatagaagcgttggaggaagcggaggcggcagcttcggcgataatctggtcaccagaagctactgatgactcgagactagtcaattggtcgacctcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 3 - protein, 646 aa, synthetic construct
  REGION 1..646: note = Synthetic Construct
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSY

SEQ ID NO: 4 - DNA, 1945 bp, synthetic construct
  misc_feature 1..1945: note = engineered nucleic acid sequence encoding Lamin A
  source 1..1945: mol_type = other DNA
atggaaacccctagccagcggagagctacaagaagcggagcccaggcctctagcacacctctgagccctaccagaatcacccggctgcaagagaaagaggacctgcaagagctgaacgaccggctggccgtgtacatcgacagagtcagaagcctggaaaccgagaacgccggcctgagactgagaatcaccgagagcgaggaagtggtgtccagagaggtgtccggaatcaaggccgcctacgaagccgaactgggcgacgccagaaagaccctggatagcgtggccaaagagcgggccagactgcagctggaactgagcaaagtgcgggaagagttcaaagaactgaaggcccggaacaccaagaaagagggcgacctgattgccgctcaggccaggctgaaggatctggaagctctgctgaacagcaaagaggccgctctgagcacagccctgagcgagaagagaacactggaaggcgagctgcacgacctgagaggacaggtggcaaaactggaagccgctctgggagaagccaagaagcagctgcaggacgagatgctgagaagagtggacgccgagaaccggctgcagaccatgaaggaagaactggacttccagaagaacatctacagcgaggaactgcgcgagacaaagcggcggcacgagacaagactggtggaaatcgacaacggcaagcagagagagttcgagagcagactggccgacgctctgcaagaactgagagcccagcacgaggaccaggtggaacagtataagaaagagctggaaaagacctacagcgccaagctggacaacgccagacagagcgccgagagaaacagcaatcttgtgggagccgctcacgaagaactgcagcagagccggatcagaatcgacagcctgtctgcccagctgtctcagctgcagaaacagctggccgccaaagaggctaagctgcgggacctcgaagattccctggccagagagagagacaccagcagaaggctgctggccgagaaagaacgcgagatggccgagatgagagcccggatgcagcagcagctggacgagtatcaagagctgctggacatcaagctggccctggacatggaaatccacgcctaccggaaactgctcgaaggcgaggaagagagactgcggctgagcccatctcctaccagccagagaagtagaggcagagcctccagccacagcagccaaacacaaggcggaggcagcgtgaccaagaagaggaagctggaaagcaccgagtccagatccagcttcagccagcacgccagaacatctggcagagtggccgtggaagaggtggacgaagagggcaaattcgtgcggctgcggaacaagagcaacgaggatcagagcatgggcaactggcagatcaagcggcagaacggcgacgatcccctgctgacctaccggttccctccaaagttcaccctgaaggccggccaggtggtcacaatttgggcagctggtgctggcgccacacactctccacctactgatctcgtgtggaaggcccagaatacctggggctgtggcaacagcctgagaactgccctgatcaacagcaccggcgaagaggtcgccatgagaaaacttgtgcggagcgtgaccgtggtggaagacgacgaggatgaggatggcgacgacctgctgcaccaccaccacggatctcactgtagcagctctggcgatcccgccgagtacaacctgagaagcagaaccgtgctgtgcggcacatgtggacagcctgccgataaggcctctgctagtggatctggcgcccaagttggcggccctatcagctctggatctagcgcctctagcgtgacagtgacccggtcctatagaagcgttggaggaagcggaggcggcagcttcggcgataatctggtcaccagaagctactgatgac

SEQ ID NO: 5 - protein, 646 aa, synthetic construct; identical to SEQ ID NO: 3
  REGION 1..646: note = amino acid sequence mature Lamin A

SEQ ID NO: 6 - DNA, 1995 bp, Homo sapiens
  source 1..1995: mol_type = other DNA; note = Homo sapiens lamin A/C (LMNA), transcript variant 1, mRNA NM_170707.4
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgtaa

SEQ ID NO: 7 - DNA, 227 bp, synthetic construct
  misc_feature 1..227: note = chicken cardiac troponin T promoter
  source 1..227: mol_type = other DNA
gggcttatatgggctcctgtgggggaagggggagcacggagggggccggggccgctgctgccaaaatagcagctcacaagtgttgcattcctctctgggcgccgggcacattcctgctgctctgcccgccccggggtgggcgccggggggaccttaaagcctctgccccccaaggagcccttcccagatagccgccggcacccaccgctccgtgggacctcggcaca

SEQ ID NO: 8 - DNA, 382 bp, synthetic construct
  misc_feature 1..382: note = CMV IE enhancer
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 9 - DNA, 127 bp, synthetic construct
  misc_feature 1..127: note = rabbit beta globin poly A (rabbit globin polyA or RBG)
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 10 - DNA, 2211 bp, adeno-associated virus hu68
  source 1..2211: mol_type = other DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 11 - DNA, 2211 bp, synthetic construct
  source 1..2211: mol_type = other DNA; note = adeno-associated virus hu68 engineered nucleic acid sequence
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 12 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 2211 bp, adeno-associated virus hu95
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagtttcaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaataatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaagccgtgggtcgttcgtccttttactgcctggaatatttcccgtcgcaaatgcttcgtaccggaaacaactttaccttcagctacgagtttgagaacgtacctttccatagcagctatgctcacagccaaagcctggaccgtctcatgaatccactcatcgaccaatacttgtactatctctcaaagactattaacggttctggacagaatcaacaaacgctaaaattcagtgtggccggacccagcaacatggcagtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcttatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagttagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 14 - DNA, 2211 bp, synthetic construct
  source 1..2211: mol_type = other DNA; note = adeno-associated virus hu95 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagcgggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccagctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 15 - protein, 736 aa, adeno-associated virus hu95
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 2211 bp, adeno-associated virus hu96
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgccgcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgagggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagttatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgcttcgtaccggaaacaactttaccttcagctacgagtttgagaacgtacctttccatagcagctacgctcacagccaaagcctggaccgactaatgaatccactcatcgaccaatacttgtactatctctcaaagactattaacggttctgggcagaatcaacaaacgctaaaattcagtgtggccggacccagcaacatggcagtccagggaagaaactacatacctggacccatctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccggtagcaacggagtcctatggacaagtggccacaaaccaccagagtgcccaagcacaggcgcaaacaggctgggttcaaaaccaaggaatacttccgggtatggtttggcaagacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 17 - DNA, 2211 bp, synthetic construct
  source 1..2211: mol_type = other DNA; note = adeno-associated virus hu96 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagagggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccatctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 18 - protein, 736 aa, adeno-associated virus hu96
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSEGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPIYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 664 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 20 - protein, 18 aa, synthetic construct
  REGION 1..18: note = Lamin A pre-protein C-terminus
LLGNSSPRTQSPQNCSIM

SEQ ID NO: 21 - protein, 6 aa, synthetic construct
  REGION 1..6: note = C-terminus motif Lamin C
VSGSRR

SEQ ID NO: 22 - protein, 80 aa, synthetic construct
  REGION 1..80: note = C-terminus motif mature Lamin A
GSHCSSSGDPAEYNLRSRTVLCGTCGQPADKASASGSGAQVGGPISSGSSASSVTVTRSYRSVGGSGGGSFGDNLVTRSY

SEQ ID NO: 23 - DNA, 618 bp, synthetic construct
  misc_feature 1..618: note = hybrid CMV IE enhancer with chicken cardiac troponin T promoter
  source 1..618: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaatagcttgggcttatatgggctcctgtgggggaagggggagcacggagggggccggggccgctgctgccaaaatagcagctcacaagtgttgcattcctctctgggcgccgggcacattcctgctgctctgcccgccccggggtgggcgccggggggaccttaaagcctctgccccccaaggagcccttcccagatagccgccggcacccaccgctccgtgggacctcggcaca

(19) United States
(12) Patent Application Publication
(10) Pub. No.: US 2025/0049949 A1
(43) Pub. Date: Feb. 13, 2025

(54) GENE THERAPY FOR LAMIN A-ASSOCIATED DEFICIENCIES

(71) Applicant: The Trustees of the University of Pennsylvania, Philadelphia, PA (US)

(72) Inventor: Christian Hinderer, Baltimore, MD (US)

(73) Assignee: The Trustees of the University of Pennsylvania, Philadelphia, PA (US)

(21) Appl. No.: 18/723,236

(22) PCT Filed: Dec. 24, 2022
(86) PCT No.: PCT/US2022/082383

§ 371 Date: Jun. 21, 2024

Related U.S. Application Data
(60) Provisional application No. 63/293,680, filed on Dec. 24, 2021.

(51) Int. Cl. A61K 48/00; A61P 9/00; C07K 14/47; C12N 15/86

(52) U.S. Cl. CPC A61K 48/0033; A61K 48/0066; A61P 9/00; C07K 14/47; C12N 15/86; C12N 2750/14143; C12N 2830/008; C12N 2830/48; C12N 2830/50

(57) ABSTRACT

Provided herein is a recombinant AAV (rAAV) comprising an AAV capsid and a vector genome packaged therein, wherein the vector genome comprises an AAV 5′ inverted terminal repeat (ITR), an expression cassette comprising an open reading frame (ORF) for a mature human Lamin A (hLaminA) under control of a regulatory sequence which direct expression of mature hLaminA in a target cell, and an AAV 3′ ITR. Also provided is a pharmaceutical composition comprising a rAAV as described herein in a formulation buffer, and a method of treatment of idiopathic dilated cardiomyopathy (DCM) or a disease associated with a mutation in a Lamin A (LMNA) gene.

### REFERENCE TO AN ELECTRONIC SEQUENCE LISTING

The electronic sequence listing filed herewith named “UPN-22-9866PCT.xml” with size of 62,136 bytes, created on date of Dec. 22, 2022, and the contents of the electronic sequence listing (e.g., the sequences and text therein) are incorporated herein by reference in entirety.

### BACKGROUND OF THE INVENTION

Idiopathic dilated cardiomyopathy (DCM) prevalence is estimated at 1:500 (Hershberger, R., Hedges, D. & Morales, A. Dilated cardiomyopathy: the complexity of a diverse genetic architecture. Nat Rev Cardiol 10, 531-547 (2013)). It was approximated that about 40% of the genetic cause for DCM stems from rare variants of less than 3 genes which in turn affect functions of various proteins, among which genes is Lamin A gene (LMNA). The estimated prevalence of mutations in Lamin A gene (LMNA) in idiopathic DCM is estimated 5.9% (19/324) as of unrelated DCM probands (United States) (Parks S B, Kushner J D, Nauman D, et al. Lamin A/C mutation analysis in a cohort of 324 unrelated patients with idiopathic or familial dilated cardiomyopathy. Am Heart J 2008; 156:161-9), and 6% (35/561) of DCM patients (Norway) (Hasselberg et al. European Heart Journal (2018) 39, 853-860). The most common loss-of-function mutations cause an adult onset-form of dilated cardiomyopathy with conduction defects. Overall estimated prevalence of LMNA-related cardiomyopathy is 1:8,000 population (40,000 patients in US). Patients typically present in the 4th or 5th decade with atrial arrhythmias or atrioventricular (AV) conduction defects, before progressing to complete AV block, dilated cardiomyopathy, ventricular arrhythmias, and end stage heart failure. By age 60 most patients have had an implantable cardioverter defibrillator placed, received a heart transplant, or died.

The Lamin A and C proteins are essential structural components of the nuclear envelope, and also play a role in gene expression through interactions with chromatin. Mutations in the Lamin A/C (LMNA) gene have also been linked to diverse clinical phenotypes other than DCM, including neuropathy, muscular dystrophy, progeria, and lipodystrophy (Kang et al. BMB Reports 2018; 51:327-37).

There is not standard treatment or cure for idiopathic DCM and LMNA-related disorders. A continuing need in the art exists for compositions and methods for effective treatment for idiopathic DCM and LMNA-related disorders.

### SUMMARY OF THE INVENTION

In one aspect, provided herein is a recombinant adeno-associated virus comprising a capsid and having packaged therein a vector genome, wherein the vector genome comprises an AAV 5′ inverted terminal repeat (ITR), an expression cassette, and AAV 3′ ITR, wherein the expression cassette comprises an engineered open reading frame (ORF) for a mature human Lamin A (hLaminA) coding sequence which encodes for mature hLaminA lacking the preprotein carboxy (C) terminus tail, wherein the ORF is operably linked to regulatory control sequences which direct expression of the mature hLaminA protein in a cell, and wherein the regulatory control sequences comprise a promoter, optionally an enhancer, and a polyadenylation (polyA) sequence. In certain embodiments, the ORF has the nucleic acid sequence of SEQ ID NO: 4 or a nucleic acid sequence at least 90% identical to SEQ ID NO: 4 which encodes mature hLamin A lacking the preprotein carboxy (C) terminal tail. In certain embodiments, the ORF is operably linked to the regulatory control sequences comprising a promoter which is a cardiac promoter. In certain embodiments, the promoter is a chicken cardiac troponin T promoter. In certain embodiment, the regulatory control sequences further comprise CMV IE enhance, rabbit globin polyadenylation sequence and/or optionally a WPRE element.

In certain embodiments, the expression cassette has the nucleic acid sequence of SEQ ID NO: 2 or a nucleic acid sequence at least 90% identical to SEQ ID NO: 2. In certain embodiments, the vector genome has the nucleic acid sequence of SEQ ID NO: 1 (CMV-IE.chTNTp.LaminA.RBG). In certain embodiments, the capsid is an AAVhu68 capsid, an AAVhu95 capsid, or AAVhu96 capsid.

In a further aspect, provided herein is a composition and pharmaceutical composition comprising a rAAV or a vector as described herein and an aqueous suspension media. In certain embodiments, the rAAV or the composition thereof is for use in the treatment of idiopathic dilated cardiomyopathy (DCM) or a disease associated with a mutation in a Lamin A (LMNA) gene. In certain embodiments, the disease associated with a mutation in a LMNA gene is selected from muscular dystrophy, neuropathy, lipodystrophy, segmental progeroid.

In another aspect, provided herein is a method for treating or ameliorating or improving one or more symptoms of an idiopathic dilated cardiomyopathy (DCM) in a subject in a need thereof. In a further aspect, provided herein is a method of treating or ameliorating or improving one or more symptoms of a disease associated with a mutation in a Lamin A (LMNA) gene in a subject. In certain embodiments, the idiopathic DCM is an early onset idiopathic DCM. In certain embodiments, the idiopathic DCM is an adult-onset form of idiopathic DCM with conduction defects. In certain embodiments, the disease associated with a loss-of-function mutation in a LMNA gene is selected from muscular dystrophy, neuropathy, lipodystrophy, segmental progeroid, optionally further selected from Emery-Dreifuss Muscular dystrophy (EDMD), Malouf syndrome (MLF), Congenital Muscular dystrophy (MDC), Limb-Gardle Muscular dystrophy type 1B (LGMDTB), Charcot-Marie-Tooth disease type 2B1 (CMT2B1), axonal neuropathy, familial partial lipodystrophy type 2 (FPLD2), Mandibuloacral dysplasia lipodystrophy (MAD), Mandibuloacral Dysplasia type A (MADA), Atypical Werner syndrome (AWS), premature aging syndrome (progeria) and Hutchinson-Gilford progeria syndrome (HGPS). In certain embodiments, the symptoms of the disease comprise atrioventricular (AV) conduction block, atrial fibrillation, atrial arrhythmia, including atrial flutter and atrial tachycardia, ventricular arrhythmias including sustained ventricular tachycardias and ventricular fibrillation (VF). IN certain embodiments, the method further comprises co-treatment with beta blockers, angiotensin-converting enzyme (ACE) inhibitors, diuretics, implantable cardioverter defibrillators (ICD), pacemakers (PM) and/or cardiac resynchronization therapy (CRT).

In another aspect, provide herein is a recombinant nucleic acid molecule comprising expression cassette of SEQ ID NO: 2. In certain embodiments, the nucleic acid molecule is a plasmid. In certain embodiments, a packaging cell is provided which comprises the expression cassette, vector genome or plasmid.

These and other aspects of the invention are apparent from the following detailed description of the invention.

### BRIEF DESCRIPTION OF THE FIGURES

FIG. 1A shows relative levels of gene transfer to NHP heart, plotted as fold change in RNA sequencing reads (prevalence of RNA reads in tissue relative to vector concentration administered) relative to AAVhu68.

FIG. 1B shows levels of transduction in mouse heart following administration with AAVhu68 or AAVhu95 comprising gene encoding for Green Fluorescent Protein, plotted as percent of GFP-positive area.

FIG. 1C shows levels of transduction in mouse heart following administration with AAVhu68 or AAVhu95 comprising gene encoding for Green Fluorescent Protein, plotted as number of copies/ng RNA.

FIG. 2A shows a Kaplan-Meier survival plot of Lamin knockout (KO) and Wild type (WT) mice administered at newborn stage with either a vehicle control or AAVhu68.hLaminA intravenously at a dose of 5×10¹⁰ GC (approximately 3×10¹³ GC/kg).

FIG. 2B shows measured body weights of Lamin knockout (KO) and Wild type (WT) mice administered at newborn stage with either a vehicle control or AAVhu68.hLaminA intravenously at a dose of 5×10¹⁰ GC (approximately 3×10¹³ GC/kg).

FIG. 2C shows a representative western blot confirming expression of LaminA in heart and lack of expression of Lamin A in liver following administration of AAVhu68.hLaminA in knock-out mice.

FIG. 3A shows a representative microscopy image from immunohistochemical (IHC) analysis of staining with anti-human lamin of FRG mouse liver tissue, following transplantation with human hepatocytes (human cells are those exhibiting lamin staining).

FIG. 3B shows a representative microscopy image from immunohistochemical (IHC) analysis of staining with anti-human lamin of mouse heart tissue, following administration at newborn stage with vehicle control.

FIG. 3C shows a representative microscopy image from immunohistochemical (IHC) analysis of staining with anti-human lamin of mouse heart tissue, following administration at newborn stage with AAVhu68.hLaminA intravenously at a dose of 5×10¹⁰ GC (approximately 3×10¹³ GC/kg).

FIG. 4A shows a representative cardiogram analysis showing RR Interval (s) over time, in Lamin A KO mice administered with AAV (0-23).

FIG. 4B shows a representative cardiogram analysis showing RR Interval (s) over time, in Lamin A KO mice administered with AAV (24-48).

FIG. 4C shows a representative cardiogram analysis showing RR Interval (s) over time in WT mice administered with vehicle (PBS) (0-12).

FIG. 4D shows a representative cardiogram analysis showing RR Interval (s) over time in WT mice administered with vehicle (PBS) (13-26).

FIG. 5A shows a representative cardiogram analysis showing RR Interval (s) over time in Lamin A KO mice administered with vehicle (PBS) (0-18).

FIG. 5B shows a representative cardiogram analysis showing RR Interval (s) over time in Lamin A KO mice administered with vehicle (PBS) (19-38).

FIG. 5C shows a representative cardiogram analysis showing RR Interval (s) over time in Lamin A KO mice administered with vehicle (PBS) (zoomed in 5A, time 0-10).

FIG. 5D shows a representative cardiogram analysis showing RR Interval (s) over time in Lamin A KO mice administered with vehicle (PBS) (zoomed in 5B, time 7.00-7.45).

FIG. 6A shows results of the echocardiogram in wild-type (WT) and knock out (KO) mice administered with vehicle (PBS) or AAV, plotted as ejection fraction (%) (one-way Anova non-parametric Kruskal-Wallis with Dunn's multiple comparison test: P<0.05, **P<0.01, ***P<0.01, ****P<0.0001).

FIG. 6B shows results of the echocardiogram in wild-type (WT) and knock out (KO) mice administered with vehicle (PBS) or AAV, plotted fractional shortening (%) (one-way Anova non-parametric Kruskal-Wallis with Dunn's multiple comparison test: P<0.05, **P<0.01, ***P<0.01, ****P<0.0001).

FIG. 6C shows results of the echocardiogram in wild-type (WT) and knock out (KO) mice administered with vehicle (PBS) or AAV, plotted as stroke volume (μL) (one-way Anova non-parametric Kruskal-Wallis with Dunn's multiple comparison test: P<0.05, **P<0.01, ***P<0.01, ****P<0.0001).

FIG. 7A shows a representative image of histology analysis of heart tissue in knock out mice following administration of PBS in knock-out mice.

FIG. 7B shows a representative image of histology analysis of heart tissue in knock out mice following administration of AAV-LMNA in knock-out mice, confirming expression of Lamin A in ventricular cardiac cells.

FIG. 8 shows a representative western blot analysis for Lamin A expression in mice administered with AAVhu95-LMNA.

FIG. 9A shows results of the LMNA telemetry study plotted as percent of WT-PBS of LMNA expression in wild-type and heterozygous knockout mice following administration with either PBS (control) or AAV-LMNA.

FIG. 9B shows results of the LMNA telemetry study plotted as percent of WT-PBS of LMNC expression in wild-type and heterozygous knockout mice following administration with either PBS (control) or AAV-LMNA.

FIG. 9C shows a representative western blot analysis of cardiac samples for Lamin A and Lamin C expression in mice (wild type and heterozygous knock-out mice) administered with AAVhu95-LMNA.

### DETAILED DESCRIPTION OF THE INVENTION

Provided herein are sequences, expression cassettes, and vector expressing human functional mature Lamin A (hLAmin A) protein and compositions containing the same. Also provided herein are methods useful for the treatment LMNA cardiomyopathy (e.g., idiopathic dilated cardiomyopathy (DCM) or a disease associated with a mutation in a Lamin A (LMNA) gene) and/or alleviating symptoms thereof. In certain embodiments, the human Lamin A (hLAmin A) protein is delivered via the AAV as provided herein.

The various nucleic acid sequences provided herein are useful for packaging functional mature hLamin A coding sequence into suitable vector (e.g., rAAV) or a genetic element useful for manufacture (e.g., plasmid).

Lamin A/C gene (LMNA) is located on chromosome 1q21.2 loci, and contains alternative splicing sites encoding for Lamin A or Lamin C proteins (Kang S., et al., 2018). The native amino acid sequences of LaminA and LaminC are identical over the first 566 amino acids, but LaminC has 6 amino acid unique-carboxy (C−) terminus “VSGSRR” (SEQ ID NO: 21)). In contrast, mature Lamin A protein has at its C-terminus:

(SEQ ID NO: 22)
“GSHCSSSGDPAEYNLRSRTVLCGTCGQPADKASASGSGAQVGGPISSG
SSASSVTVTRSYRSVGGSGGGSFGDNLVTRSY”.

SEQ ID NO: 19 provides the full-length Lamin A pre-protein, i.e., 664 amino acids, which is the mature Lamin A with the above carboxy terminus and an 18 amino acid carboxy tail comprising a CaaX motif (i.e., CSIM) and farnesylated motif in a carboxy (C)-terminus of the amino acid sequence. The pre-Lamin A maturates by protease-mediated cleavage of the last 18 amino acid, resulting in a mature Lamin A. SEQ ID NO: 5 refers to the mature Lamin A protein, which lacks the pre-protein C-terminus tail motifs

(LLGNSSPRTQSPQNCSIM; SEQ ID NO: 20).

As used herein, the term “functional Lamin A” and/or “functional mature human Lamin A” refers to a protein having an amino acid sequence of the mature Lamin A protein having the sequence of SEQ ID NO: 5 or a sequence about 95% to about 100% identical thereto, at least 95%, at least 96%, at least 97%, at least 98%, at least 99%, 99.9% identical thereto, and values therebetween, as determined over contiguous amino acid sequences which provide at least 10%, at least 20%, at least 30%, at least 40%, at least 50%, at least 60%, at least 70%, at least 75%, at least 80%, at least 90%, or a similar and/or same, or greater than 100% biological activity or function as a wild type mature human Lamin A. This biological activity or function may be determined by any suitable means, e.g., in an in vitro assay, animal model or by monitoring patients post-treatment for correction of symptoms of a condition associated with dysfunctional or non-functional LaminA. In certain embodiments, the mutant mature human LaminA may have one or more conservative amino acid substitutions as compared to an amino acid sequence of SEQ ID NO: 5, e.g., 1 to 30 amino acid changes. In certain embodiments, a mutant mature human LaminA protein may be about 95% to about 100% identical to SEQ ID NO: 5 and comprise one or more conservative, non-conservative amino acid substitutions, as well as insertions and/or deletions. In certain embodiments, substitutions which result in a mutant human LaminA having SEQ ID NO: 21 (“VSGSRR”) in the region of amino acids 567 to 572 (as referenced to SEQ ID NO: 5) are excluded. In certain embodiments, about 10% to about 100% of wild type mature human Lamin. In certain embodiments, at least 10%, at least 20%, at least 30%, at least 40%, at least 50%, at least 60%, at least 70%, at least 75%, at least 80%, at least 90%, or at least 95% of normal wild type mature human Lamin A activity and/or function is achieved. In certain embodiments, greater than 100%, e.g., about 105%, about 110%, about 115%, about 120%, about 125%, about 130%, or greater of normal wild type mature human Lamin A activity and/or function is achieved.

As described herein, any variant or mutant of mature human Lamin A expressed from nucleic acid sequences as provided herein or variations thereof, which restores a desired function, ameliorates a symptom, improves symptoms associated with DCM or a disease associated with a mutation in LMNA gene. The amino acid substitutions are selected to avoid any changes which render the mature human Lamin A dysfunctional or non-functional, e.g., by introducing substitutions associated with disease as described herein.

As used herein, the “conservative amino acid replacement” or “conservative amino acid substitutions” refers to a change, replacement or substitution of an amino acid to a different amino acid with similar biochemical properties (e.g., charge, hydrophobicity and size), which is known by practitioners of the art. Also see, e.g., FRENCH et al. What is a conservative substitution?Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety. Without wishing to be bound by theory, the conservative amino acid replacement excludes the amino acid substitutions to the mature Lamin A protein which is/are associated with a disease, as described herein.

The following are examples of amino acid substitutions which may render a mature human LaminA dysfunctional or non-functional. For example, amino acid substitutions in Lamin A which are associated with DCM may include one or more of Q6X, E203K, R25G, R25P, E203G, R25W, L215P, L59R, R225X, R60G, Y267C, E82K, E317K, L85R, A347K, R89L, R349L, K97E, Q355X, S143P, R399C, E161K, R435C, R190W, R541C, D192G, R541S, N195K, S573L, S573L, R133P, E358K, L530P, R584H, T623S, and R644C (as referenced numbering of the amino acid sequence of Lamin A pre-protein; SEQ ID NO: 19). Amino acid substitutions in Lamin A protein which are associated with muscular dystrophy may include one or more of: Q6X, G232E, G449D, A57P, N39S, R25G, R25P, L248P, R453W, L59R, R50P, Y259X, R25G, R249Q, L454P, R249W, E358K, E33G, R249W, N4561, L302P, R377H, L35V, F260L, N456K, E358K, R377L, N39S, Y267C, D461Y, L380S, R399C, A43T, S268P, W467R, R453P, Y481H, Y45C, L271P, I469T, R455P, R50S, Q294P, W520S, N456D, I63S, S295P, R527P, I63N, S303P, T528K, E65G, R336Q, T528R, R89C, R343Q, L529P, R133P, E358K, L530P, L140P, E361K, R541H, T150P, M371K, R541S, R189P, R377L, R541P, R190Q, R386K, G602S, R196S, R401C, R624H, H222P, V442A, H222Y, D446V (as referenced numbering of the amino acid sequence of Lamin A pre-protein; SEQ ID NO: 19). Amino acid substitutions in Lamin A protein which are associated with neuropathy may include R298C (as referenced numbering of the amino acid sequence of Lamin A pre-protein; SEQ ID NO: 19). Amino acid substitutions in Lamin A protein which are associated with lipodystrophy may include one or more of: R25W, V440M, R60G, R471C, R62G, R527C, AK208, R527H, D230N, A529V, G456D, R482W, R482Q, R482L, P485R, K486N, S573L, R582H, R584H (as referenced numbering of the amino acid sequence of Lamin A pre-protein; SEQ ID NO: 19). Amino acid substitutions in Lamin A protein which are associated with segmental progeroid may include one or more of: A57P, T10I, R133L, S143E, L140R, S143F, D300N, E145K, Q656Q, R471C, R527C, T528M, M540T, K542N, E578V, V607V, G608S, G608G, T623S (as referenced numbering of the amino acid sequence of Lamin A pre-protein; SEQ ID NO: 19). See also, Kang, S., et al., Laminopathies; Mutations on single gene and various human genetic diseases, BMB Reports 2018, 51(7):327-337; Rankin, J., et al., The laminopathies: a clinical review, Clin. Genet., 2006, 70:261-274; Vigouroux C, Bonne G. Laminopathies: One Gene, Two Proteins, Five Diseases. In: Madame Curie Bioscience Database [Internet]. Austin (TX): Landes Bioscience; 2000-2013, ncbi.nlm.nih.gov/books/NBK6151/, which are all incorporated herein by reference in its entirety.

In one embodiment, the functional mature hLamin A has an amino acid sequence of SEQ ID NO: 5 or an amino acid sequence at least about 95% (e.g., at least 95%, 96%, 97%, 98%, 99%, or 99.9%) identical thereto.

In certain embodiments, a functional mature hLamin A protein ameliorates symptoms or delays progression of LMNA cardiomyopathy (e.g., idiopathic dilated cardiomyopathy (DCM)) or a disease associated with a mutation in a Lamin A (LMNA) gene in an animal model. One exemplified animal model is a LMNA-knock out (LMNA-ko) mouse. Other suitable models may be used. The LMNA cardiomyopathy symptoms or progression may be evaluated using various assays/methods, including but not limited to, a survival plot (e.g., Kaplan-Meier survival plot), monitoring body weights, echocardiogram (echo) and electrocardiogram (EKG or ECG). In certain embodiment, administration or expression of a functional mature hLamin A protein in an animal model leads to amelioration of LMNA cardiomyopathy symptoms or delay in LMNA cardiomyopathy progression shown by an assay result which is at least about 10%, 20%, 30%, 40%, 50%, 60%, 70%, 80%, 90%, 95%, 100%, or more than 100% of that obtained in a corresponding wildtype animal. In certain embodiment, administration or expression of a functional mature hLamin A protein in a LMNA cardiomyopathy animal model leads to amelioration of LMNA cardiomyopathy symptoms or delay in LMNA cardiomyopathy progression shown by an improved assay result which is at least about 10%, 20%, 30%, 40%, 50%, 60%, 70%, 80%, 90%, 95%, 100%, or more than 100% of that obtained from a corresponding non-treated LMNA cardiomyopathy animal.

### LaminA Open Reading Frame and Nucleic Acid Molecules

In one aspect, provided herein is a hLamin A coding sequence which is an engineered hLamin A coding sequence. In one embodiment, the engineered sequence is useful to improve production, transcription, expression or safety in a subject. In another embodiment, the engineered sequence is useful to increase efficacy of the resulting therapeutic compositions or treatment. In a further embodiment, the engineered sequence is useful to increase the efficacy of the functional nature hLamin A protein being expressed, but may also permit a lower dose of a therapeutic reagent that delivers the functional protein to increase safety.

In one aspect, provided herein is a recombinant nucleic acid molecule comprising an engineered hLamin A coding sequence which encodes a functional mature human Lamin A (hLamin A). In certain embodiments, the engineered hLamin A coding sequence comprises a nucleic acid sequence of SEQ ID NO: 4 or a sequence of about 90%, at least 95% identical, at least 97% identical, at least 98% identical, or 99% to 100% identical to SEQ ID NO: 4 and which expresses the functional mature hLamin A protein.

In certain embodiments, the engineered hLaminA coding sequence is SEQ ID NO: 4 or a nucleic acid sequence at least 90% (e.g., at least 91%, at least 92%, at least 93%, at least 94%, at least 95%, at least 96%, at least 97%, at least 98%, at least 99%, or at least 99.9%) identical thereto which encodes an amino acid sequence of SEQ ID NO: 5.

A “nucleic acid”, as described herein, can be RNA, DNA, or a modification thereof, and can be single or double stranded, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “Clustal Omega” “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “Clustal Omega”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

Nucleic acid sequences described herein can be cloned using routine molecular biology techniques, or generated de novo by DNA synthesis, which can be performed using routine procedures by service companies having business in the field of DNA synthesis and/or molecular cloning (e.g., GeneArt, GenScript, Life Technologies, Eurofins). The nucleic acid sequences encoding the miRNA or modified snRNA described herein are assembled and placed into any suitable genetic element, e.g., naked DNA, phage, transposon, cosmid, episome, etc., which transfers the sequences carried thereon to a host cell, e.g., for generating non-viral delivery systems (e.g., RNA-based systems, naked DNA, or the like), or for generating viral vectors in a packaging host cell, and/or for delivery to a host cells in a subject. In one embodiment, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

It should be understood that the hLamin A coding sequences described herein are intended to be applied to other compositions, regiments, aspects, embodiments and methods described across the Specification.

### Expression Cassette and Vector Genome

Provided herein is a nucleic acid sequence comprising the engineered hLamin A coding sequence under control of regulatory sequences which direct the functional mature hLamin A expression in a target cell, also termed as an expression cassette. In certain embodiments, the expression cassette comprises an open reading frame (ORF) for a functional mature hLamin A coding sequence which encode functional mature hLamin A lacking preprotein carboxy (C) terminus tail, wherein the ORF is operably linked to regulatory control sequences which direct expression of the mature functional hLamin A protein in a cell, and wherein the regulatory control sequences comprise a promoter, or a hybrid promoter, optionally an enhancer, and a polyadenylation (polyA) sequences.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g., a different copy number, or under the control of different regulatory elements.

The expression cassette may contain regulatory sequences upstream (5′ to) of the gene sequence, e.g., one or more of a promoter, a hybrid promoter, an enhancer, an intron, etc., and one or more of an enhancer, or regulatory sequences downstream (3′ to) a gene sequence, e.g., 3′ untranslated region (3′ UTR) comprising a polyadenylation (polyA) site, among other elements.

In certain embodiments, the regulatory sequences comprise one or more of a promoter, an enhancer, an intron, a transcription factor, a transcription terminator, an efficient RNA processing signals such as splicing and polyadenylation site signals (polyA), a sequences that stabilize cytoplasmic mRNA, for example Woodchuck Hepatitis Virus (WHP) Posttranscriptional Regulatory Element (WPRE), and sequences that enhance translation efficiency (i.e., Kozak consensus sequence). In certain embodiments the selected promoter is a constitutive promoter. In certain embodiments, the promoter is a ubiquitous promoter. For example such promoters may include chicken beta-actin (CB) promoter, hybrid of a cytomegalovirus immediate-early enhancer and the chicken R-actin promoter (a CB7 promoter), human cytomegalovirus (CMV) promoter, ubiquitin C promoter (UbC), the early and late promoters of simian virus 40 (SV40), U6 promoter, metallothionein promoters, EFlu promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the R-actin promoter (Lai et al., Proc. Natl. Acad. Sci. USA 86: 10006-10010 (1989)), the long terminal repeats (LTR) of Moloney Leukemia Virus and other retroviruses, the thymidine kinase promoter of Herpes Simplex Virus and other constitutive promoters known to those of skill in the art.

In certain embodiments, the promoter is a tissue- or cell specific-promoter. In certain embodiments, the promoter is cardiac specific promoter, e.g., cardiac troponin T (cTNT), desmin (DES), alpha-myosin heavy chain (α-MHC), myosin light chain 2 (MLC-2) promoters. See also, Pacak, C. A., et al., Tissue specific promoters improve specificity of AAV9 mediated transgene expression following intra-vascular gene delivery in neonatal mice, Genetic Vaccines and Therapy 2008, 6:13. In certain embodiments, the expression cassette comprises a promoter which is a chicken cardiac Troponin T promoter (also referred to as chicken TnT or chTnT). In certain embodiments, the chTnT promoter comprises nucleic acid sequence of SEQ ID NO: 7. In certain embodiments, the promoter is a hybrid promoter. In certain embodiments, the promoter is a hybrid cardiac promoter. As used herein, the term “hybrid promoter” refers to a regulatory control sequence comprising a hybrid between an enhancer, a spacer sequence, and a promoter sequence. In certain embodiments, the hybrid cardiac promoter comprises a CMV IE enhancer sequence, a spacer sequence, a chicken cardiac troponin T promoter. In certain embodiments, the spacer sequence is less than 100% identical to the sequence in the examples herein. In certain embodiments, the spacer sequence comprises at least two (2) to at least ten (10) nucleotides. In certain embodiments, the spacer sequence is at least nine (9) nucleotides. In certain embodiments, the spacer comprises nucleic acid sequence “CAATAGCTT”. In certain embodiments, the spacer sequence comprises nucleic acid sequence “CA”. In certain embodiments, the spacer sequence is selected so that it does not encode any protein, peptide or vector genome element. See also, U.S. Provisional Patent Application No. 63/293,678, filed Dec. 24, 2021, which is incorporated herein by reference in its entirety.

In certain embodiments, the hybrid cardiac promoter comprises nucleic acid sequence of SEQ ID NO: 23. In certain embodiment, the hybrid cardiac promoter comprises nucleic acid sequence at least 99% identical to SEQ ID NO: 23. The variations in the nucleic acid sequence of the hybrid cardiac promoter includes substitutions of nucleotides in the spacer sequence, and optionally include insertion and deletion of nucleotides in spacer sequence.

In one embodiment, expression of the gene product is controlled by a regulatable promoter that provides tight control over the transcription of the sequence encoding the gene product, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. These response elements may include, a hypoxia response element (HRE) that binds HIF-Ia and R, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991).

Using such promoters, expression of the transgene can be controlled, for example, by the Tet-on/off system (Gossen et al., 1995, Science 268:1766-9; Gossen et al., 1992, Proc. Natl. Acad. Sci. USA., 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA., 91(17):8180-4; Schillinger et al., 2005, Proc. Natl. Acad. Sci. USA. 102(39):13789-94); and the humanized tamoxifen-dep regulatable system (Roscilli et al., 2002, Mol. Ther. 6(5):653-63).

In another aspect, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Plasmid Kit, Takara Bio iDimerize regulated transcription kit, or comparable kits from QuantiTect, Sensiscript, or the like, each of which is incorporated herein by reference in its entirety. These systems are designed to be induced by rapamycin or one of its analogs, referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck]. In certain embodiments, rapamycin or a suitable analog may be delivered locally or systemically to the AAV-transfected cells.

In certain embodiments, the expression cassette comprises one or more expression enhancers. In one embodiment, the expression cassette contains two or more expression enhancers. These enhancers may be the same or may differ from one another. In certain embodiments, the enhancer is a cytomegalovirus immediate early enhancer (CMV IE enhancer). In certain embodiments, the CMV IE enhancer comprises nucleic acid of SEQ ID NO: 8. In certain embodiments, the enhancer is a cardiac enhancer. In certain embodiments, the cardiac enhancer is chicken troponin T enhancer. In certain embodiments, the enhancer is a rat α-myosin heavy enhancer. This/these enhancer/s may be present in two copies which are located adjacent to one another. Alternatively, the dual copies of the enhancer may be separated by one or more sequences. In a further embodiment, the enhancer(s) is selected from one or more of an APB enhancer, an ABPS enhancer, an alpha mic/bik enhancer, a TTR enhancer, an en34 enhancer, an ApoE enhancer, a CMV enhancer, or an RSV enhancer. In yet another embodiment, the regulatory elements comprise an intron. In a further embodiment, the intron is selected from chicken beta actin intron (CBA), human beta globin, IVS2, SV40 (Promega), bGH, alpha-globulin, beta-globulin, collagen, ovalbumin, or p53. See, e.g., WO 2011/126808. In one embodiment, the regulatory elements comprise a polyA. In a further embodiment, the polyA is a synthetic polyA or from bovine growth hormone (bGH), human growth hormone (hGH), SV40, rabbit β-globin (RBG), or modified RBG (mRBG). Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619.

In certain embodiments, the expression cassettes may include one or more expression enhancers such as post-transcriptional regulatory element from hepatitis viruses of woodchuck (WPRE), human (HPRE), ground squirrel (GPRE) or arctic ground squirrel (AGSPRE); or a synthetic post-transcriptional regulatory element. These expression-enhancing elements are particularly advantageous when placed in a 3′ UTR and can significantly increase mRNA stability and/or protein yield. In certain embodiments, the expressions cassettes provided include a regulator sequence that is a woodchuck hepatitis virus posttranscriptional regulatory element (WPRE) or a variant thereof. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene. See also, Kingsman S. M., Mitrophanous K., & Olsen J. C. (2005), Potential Oncogene Activity of the Woodchuck Hepatitis Post-Transcriptional Regulatory Element (Wpre).” Gene Ther. 12(1):3-4; and Zanta-Boussif M. A., Charrier S., Brice-Ouzet A., Martin S., Opolon P., Thrasher A. J., Hope T. J., & Galy A. (2009), Validation of a Mutated Pre Sequence Allowing High and Sustained Transgene Expression While Abrogating Whv-X Protein Synthesis: Application to the Gene Therapy of Was, Gene Ther. 16(5):605-19, both of which are incorporated herein by reference in its entirety. In other embodiments, enhancers are selected from a non-viral source. In certain embodiments, no WPRE sequence is present.

In certain embodiments, the expression cassette comprises regulatory control sequences comprising a cardiac promoter. In certain embodiments, the expression cassette comprises regulatory control sequences comprising a cardiac troponin T (cTnT) promoter. In certain embodiments, the expression cassette comprises regulatory control sequences comprising a hybrid cardiac promoter comprising a CMV IE enhancer, a spacer sequence, and a chicken cardiac Troponin T promoter (chTnT) with. In certain embodiments, the expression cassette comprises chTnT comprising nucleic acid sequence of SEQ ID NO: 7 with CMV IE enhancer comprising nucleic acid sequence of SEQ ID NO: 8. In certain embodiments, the expression cassette comprises the hybrid cardiac promoter comprising nucleic acid sequence of SEQ ID NO: 23 or a sequence at least 99% identical to SEQ ID NO: 23. In certain embodiments, the regulatory control sequences comprise polyA sequence which is a rabbit beta globin polyA sequence. In certain embodiments, the expression cassette comprises rabbit beta globin polya sequence comprising nucleic acid sequence of SEQ ID NO: 9. In certain embodiments, the expression cassette comprises chTnT promoter—optionally with CMV IE enhancer—hLamin A coding sequence—rabbit beta-globin polyA. In certain embodiments, the expression cassette comprises, from 5′ to 3′, CMV IE enhancer, chTnT promoter, hLamin A coding sequence, and rabbit beta globin polyA. In certain embodiments, the expression cassette comprises nucleic acid sequence of SEQ ID NO: 2, or a sequence 90% identical to SEQ ID NO: 2. In certain embodiments, the expression cassette comprises nucleic acid sequence at least 95%, at least 96%, at least 97%, at least 98%, at least 99 to at least 100% identical to SEQ ID NO: 2.

In certain embodiments, the expression cassette is CMVe.chTNTp.Lamin.RBG which comprises nucleic acid sequence of SEQ ID NO: 2.

In certain embodiments, the expression cassette comprising hLamin A coding sequence and may include other regulatory sequences therefor. The regulatory sequences necessary are operably linked to the hLamin A coding sequence in a manner which permits its transcription, translation and/or expression in target cell.

In a further aspect, provided herein is a vector genome comprising an AAV 5′ inverted terminal repeat (ITR), an expression cassette, and an AAV3′ ITR, wherein the expression cassette comprises a nucleic acid sequence encoding a functional mature hLamin A gene operably linked to expression control sequences which direct expression thereof in a cell comprising the selected gene.

In certain embodiments, the vector genome comprises an engineered nucleic acid sequence comprising open reading frame (ORF) for a functional mature hLamin A coding sequence which encode functional mature hLamin A lacking preprotein carboxy (C) terminus tail, wherein the ORF is operably linked to regulatory control sequences which direct expression of the mature functional hLamin A protein in a cell, and wherein the regulatory control sequences comprise a promoter, optionally an enhancer, and a polyadenylation (polyA) sequences.

In certain embodiments, the vector genome comprises an expression cassette having a nucleic acid sequence of SEQ ID NO: 2 or a sequence at least about 90% identical to SEQ ID NO: 2. In certain embodiments, the vector genome comprises a nucleic acid molecule comprising, 5′ to 3′, AAV5′ ITR—hybrid cardiac promoter—engineered hLamin A coding sequence—rabbit beta globin polyA—AAV3′ ITR. In certain embodiments, the vector genome comprises a nucleic acid molecule comprising, 5′ to 3′, AAV5′ ITR—optionally CMV IE promoter—optionally spacer sequence—chTnT promoter—engineered hLamin A coding sequence—rabbit beta globin polyA—AAV3′ ITR. In certain embodiments, the vector genome comprises a nucleic acid molecule comprising, 5′ to 3′, AAV5′ ITR—CMV IE promoter—spacer sequence—chTnT promoter—engineered hLamin A coding sequence—rabbit beta globin polyA—AAV3′ ITR. In certain embodiments, the vector genome comprises nucleic acid sequence of SEQ ID NO: 1. In certain embodiments, the vector genome comprises nucleic acid sequence of at least 95%, at least 96%, at least 97%, at least 98%, at least 99 to at least 100% identical to SEQ ID NO: 1.

In certain embodiments, the vector genome is 5′ITR.CMVe.chTNTp.Lamin.RBG.3′ITR comprising nucleic acid sequence of SEQ ID NO: 1, wherein the “CMVe.chTNTp” refers to the hybrid cardiac promoter comprising a CMV IE enhancer, a spacer sequence and a chicken cardiac troponin T promoter.

In certain embodiments, the target cell is cardiac tissue cell. In certain embodiments, the target cell is heart cell. In certain embodiments, the target cell is any other cell which expresses a functional mature Lamin A protein in a subject without idiopathic DCM or a disease associated with a mutation in a LMNA gene.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR (also referred to as 5′ ITR), coding sequence(s) (i.e., transgene(s)), and an AAV 3′ ITR (also referred to as 3′ ITR). ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding hLamin A operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids. In certain embodiments, the vector genome is an expression cassette having inverted terminal repeat (ITR) sequences necessary for packaging the vector genome into the AAV capsid at the extreme 5′ and 3′ end and containing therebetween a hLaminA gene as described herein operably linked to sequences which direct expression thereof. In certain embodiments, a vector genome may comprise at a minimum from 5′ to 3′, an AAV 5′ ITR, coding sequence(s), and an AAV 3′ ITR. In certain embodiments, the ITRs are from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

It should be understood that the compositions in the expression cassette and vector genomes described herein are intended to be applied to other compositions, regiments, aspects, embodiments and methods described across the Specification.

### Vector

In one aspect, provided herein is a vector comprising an engineered open reading frame (ORF) for mature human Lamin A (hLaminA), wherein the ORF has a mature hLaminA coding sequence, which is a nucleic acid sequence encoding a functional mature human Lamin A (hLaminA) lacking the preprotein carboxy (C) terminus tail, wherein ORF is operably linked to regulatory control sequences which direct the expression of the mature hLaminA in a cell, wherein the hLaminA coding sequence comprises nucleic acid sequence of SEQ ID NO: 4 or a nucleic acid sequence at least 90% identical to SEQ ID NO: 4 which encode amino acid sequence of SEQ ID NO: 5, and wherein the regulatory control sequences include a cardiac specific promoter.

In certain embodiments, the vector comprises hLamin A coding sequence comprising a nucleic acid sequence of SEQ ID NO: 4 or a sequence of at least 90%, at least 95% identical, at least 97% identical, at least 98% identical, or 99% to 100% identical to SEQ ID NO: 4 and which expresses the functional mature hLamin A protein. In certain embodiments, the vector comprises hLamin A coding sequence comprising a nucleic acid sequence of SEQ ID NO: 4 or a nucleic acid sequence at least 90% (e.g., at least 91%, at least 92%, at least 93%, at least 94%, at least 95%, at least 96%, at least 97%, at least 98%, at least 99%, or at least 99.9%) identical thereto which encodes an amino acid sequence of SEQ ID NO: 5.

A “vector” as used herein is a biological or chemical moiety comprising a nucleic acid sequence which can be introduced into an appropriate target cell for replication or expression of said nucleic acid sequence. Examples of a vector includes but not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule into which an exogenous or heterologous or engineered nucleic acid encoding a functional SGSH may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In one embodiment, the vector is a non-viral plasmid that comprises an expression cassette described thereof, e.g., “naked DNA”, “naked plasmid DNA”, naked RNA, and mRNA; coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. See, e.g., X. Su, et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiments, the vector described herein is a “replication-defective virus” or a “viral vector” which refers to a synthetic or artificial viral particle in which an expression cassette containing a nucleic acid sequence encoding hLamin A is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the nucleic acid sequence encoding hLamin A flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant virus vector is an adeno-associated virus (AAV), an adenovirus, a bocavirus, a hybrid AAV/bocavirus, a herpes simplex virus or a lentivirus.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a heart cell, or a stem cell.

It should be understood that the compositions in the vector described herein are intended to be applied to other compositions, regiments, aspects, embodiments and methods described across the Specification.

Recombinant Adeno-associated Virus (rAAV)

Provided herein is a recombinant adeno-associated virus (rAAV) useful for treating idiopathic dilated cardiomyopathy or a disease associated with a dysfunctional LMNA gene, e.g., such as caused by a complete or partial loss-of-function mutation. The rAAV comprises (a) an AAV capsid; and (b) a vector genome packaged in the AAV capsid of (a). Suitably, the AAV capsid selected targets the cells to be treated. In certain embodiments, the capsid is from Clade F. However, in certain embodiments, another AAV capsid source may be selected, i.e., Clade A. In certain embodiments, the AAV capsid is AAVhu68 capsid. In certain embodiments, the AAV capsid is AAVhu95 capsid. In certain embodiments, the AAV capsid is AAVhu96 capsid. The vector genome comprises an AAV 5′ inverted terminal repeat (ITR), an engineered nucleic acid sequence encoding a functional mature hLamin A as described herein, a regulatory sequence which direct expression of hLamin A in a target cell, and an AAV 3′ ITR.

In one aspect, the rAAV.hLaminA is for use in the treatment of idiopathic DCM. In certain embodiments, the rAAV.hLaminA is for the use in treatment early-onset idiopathic DCM. In certain embodiments, the rAAV.hLaminA is for the use in treatment adult-onset idiopathic DCM. In certain embodiments, the rAAV comprises a vector genome comprising 5′ AAV ITR, an expression cassette, and 3′ AAV ITR, wherein the expression cassette comprises an engineered nucleic acid sequence comprising open reading frame (ORF) for a functional mature hLamin A coding sequence which encode functional mature hLamin A lacking preprotein carboxy (C) terminus tail, wherein the ORF is operably linked to regulatory control sequences which direct expression of the mature functional hLamin A protein in a cell, and wherein the regulatory control sequences comprise a promoter, optionally an enhancer, and a polyadenylation (polyA) sequences (rAAV.hLaminA). In certain embodiments, the rAAV comprises the vector genome comprising an expression cassette having a nucleic acid sequence of SEQ ID NO: 2 or a sequence at least about 90% identical to SEQ ID NO: 2. In certain embodiments, the rAAV comprises vector genome comprising a nucleic acid molecule comprising, 5′ to 3′, AAV5′ ITR—hybrid cardiac promoter—engineered hLamin A coding sequence—rabbit beta globin polyA—AAV3′ ITR. In certain embodiments, the rAAV comprises vector genome comprising a nucleic acid molecule comprising, 5′ to 3′, AAV5′ ITR—optionally CMV IE promoter—optionally spacer sequence—chTnT promoter—engineered hLamin A coding sequence—rabbit beta globin polyA—AAV3′ ITR. In certain embodiments, the rAAV comprises a vector genome comprising a nucleic acid molecule comprising, 5′ to 3′, AAV5′ ITR—CMV IE promoter —spacer sequence—chTnT promoter—engineered hLamin A coding sequence—rabbit beta globin polyA—AAV3′ ITR. In certain embodiments, the rAAV comprises a vector genome comprising nucleic acid sequence of SEQ ID NO: 1 or a sequence of at least 95%, at least 96%, at least 97%, at least 98%, at least 99 to at least 100% identical to SEQ ID NO: 1 (rAAV.CMV-IE.chTNTp.LaminA.RBG).

In certain embodiments, the AAV capsid for the compositions and methods described herein is chosen based on the target cell. In certain embodiment, the AAV capsid transduces a heart cell. In certain embodiments, other AAV capsid may be chosen.

In certain embodiments, the Clade F AAV capsid is an AAVhu68 capsid [See, e.g., US2020/0056159; PCT/US21/55436; SEQ ID NO: 10 and 11 for nucleic acid sequence; SEQ ID NO: 12 for amino acid sequence], an AAVhu95 capsid [See, e.g., U.S. Provisional Application No. 63/251,599, filed Oct. 2, 2201; SEQ ID NOs: 13 and 14 (hu95 nucleic acid sequence) and SEQ ID NO: 15 (hu95 amino acid sequence), an AAVhu96 capsid [See, e.g., U.S. Provisional Application No. 63/251,599, filed Oct. 2, 2201; SEQ ID NOs: 16 and 17 (hu96 nucleic acid sequence) and SEQ ID NO: 18 (hu96 amino acid sequence), or AAV9 [See, e.g., U.S. Pat. No. 7,906,111] or engineered mutants and variants thereof [see, e.g., WO2020/200499; WO2003/054197]. See also, International Patent Application No. PCT/US2022/077315, filed Sep. 30, 2022, which is incorporated herein by reference in its entirety.

In certain embodiments, the AAV capsid is a non-clade F capsid, for example a Clade A, B, C, D, or E capsid. In certain embodiment, the non-Clade F capsid is an AAV1 or a variation thereof. In certain embodiment, the AAV capsid transduces a target cell other than the heart cells. In certain embodiments, the AAV capsid is a Clade A capsid (e.g., AAV1, AAV6, AAVrh91), a Clade B capsid (e.g., AAV 2), a Clade C capsid (e.g., hu53), a Clade D capsid (e.g., AAV7), or a Clade E capsid (e.g., rh10).

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. The term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine—glycine pairs and optionally further comprising other deamidated amino acids, wherein the deamidation results in an amino acid change and other optional modifications.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

As used herein, the terms “target cell” and “target tissue” can refer to any cell or tissue which is intended to be transduced by the subject AAV vector or in which expression of hLamin A is desired. The term may refer to any one or more of muscle, liver, lung, airway epithelium, central nervous system, neurons, eye (ocular cells), or heart. In certain embodiments, the term “target cell” is intended to reference the cells of the subject being treated for idiopathic Lamin A or a disease associated with a mutation in a LMNA gene. In certain embodiments, the vector is delivered to a target cell ex vivo. In certain embodiments, the vector is delivered to the target cell in vivo.

Additionally, provided herein, is an rAAV production system useful for producing a rAAV as described herein. The production system comprises a cell culture comprising (a) a nucleic acid sequence encoding an AAV capsid protein; (b) the vector genome; and (c) sufficient AAV rep functions and helper functions to permit packaging of the vector genome into the AAV capsid. In certain embodiments, the vector genome is SEQ ID NO: 1. In certain embodiments, the cell culture is a human embryonic kidney 293 cell culture. In certain embodiments, the AAV rep is from a different AAV. In certain embodiments, wherein the AAV rep is from AAV2. In certain embodiments, the AAV rep coding sequence and cap genes are on the same nucleic acid molecule, wherein there is optionally a spacer between the rep sequence and cap gene.

For use in producing an AAV viral vector (e.g., a recombinant (r) AAV), the vector genomes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

In certain embodiments, a plasmid useful in producing an rAAV particle is provided which comprises a vector genome comprising a AAV 5′ ITR, an expression cassette, and a AAV3′ ITR, wherein expression cassette comprises the engineered nucleic acid sequence comprising open reading frame (ORF) for a functional mature hLamin A coding sequence which encode functional mature hLamin A lacking preprotein carboxy (C) terminus tail, wherein the ORF is operably linked to regulatory control sequences which direct expression of the mature functional hLamin A protein in a cell, and wherein the regulatory control sequences comprise a promoter, optionally an enhancer, and a polyadenylation (polyA) sequences. In certain embodiments, a nucleic acid (e.g., a plasmid) useful in rAAV production comprises a vector genome comprising a AAV5′ ITR, a hybrid cardiac promoter (comprising a CMV IE enhancer, a spacer sequence, and a chTnT promoter), a function mature hLamin A coding sequence, a rabbit beta globin polyA sequence, and a AAV3′ ITR. In certain embodiments, a nucleic acid (e.g., a plasmid) useful in rAAV production comprises a vector genome comprising nucleic acid sequence of SEQ ID NO: 1.

Methods for generating and isolating AAVs suitable for use as vectors are known in the art. See generally, e.g., Grieger & Samulski, 2005, Adeno-associated virus as a gene therapy vector: Vector development, production and clinical applications, Adv. Biochem. Engin/Biotechnol. 99: 119-145; Buning et al., 2008, Recent developments in adeno-associated virus vector technology, J. Gene Med. 10:717-733; and the references cited below, each of which is incorporated herein by reference in its entirety. As used herein, a gene therapy vector refers to a rAAV as described herein, which is suitable for use in treating a patient. For packaging a gene into virions, the ITRs are the only AAV components required in cis in the same construct as the nucleic acid molecule containing the gene. The cap and rep genes can be supplied in trans.

In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV having a capsid selected from AAVhu68, AAVhu95 or AAVhu96 is provided. Such a cell culture contains a nucleic acid which expresses the AAVhu68 capsid protein in the host cell (e.g., SEQ ID NO: 10 or SEQ ID NO: 11; a nucleic acid molecule suitable for packaging into the AAVhu68 capsid, e.g., a vector genome which contains AAV ITRs and a non-AAV nucleic acid sequence encoding a gene operably linked to regulatory sequences which direct expression of the gene in a host cell; and sufficient AAV rep functions and adenovirus helper functions to permit packaging of the vector genome into the recombinant AAVhu68, or AAVhu95 capsid (e.g., SEQ ID NO: 13 or SEQ ID NO: 14), AAVhu96 capsid (e.g., SEQ ID NO: 16 or SEQ ID NO: 17). In one embodiment, the cell culture is composed of mammalian cells (e.g., human embryonic kidney 293 cells, among others) or insect cells (e.g., Spodoptera frugiperda (Sf9) cells). In certain embodiments, baculovirus provides the helper functions necessary for packaging the vector genome into the recombinant AAVhu68, AAVhu95 or AAVhu96 capsid.

Optionally the rep functions are provided by an AAV other than AAV2, selected to complement the source of the ITRs.

In one embodiment, cells are manufactured in a suitable cell culture (e.g., HEK 293 or Sf9) or suspension. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV vector genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, Adenovirus-adeno-associated virus hybrid for large-scale recombinant adeno-associated virus production, Human Gene Therapy 20:922-929, the contents of each of which is incorporated herein by reference in its entirety. Methods of making and using these and other AAV production systems are also described in the following US patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. An affinity chromatography purification followed anion exchange resin chromatography are used to purify the vector drug product and to remove empty capsids. These methods are described in more detail in WO2017/160360, filed Dec. 9, 2016, entitled “Scalable Purification Method for AAV9”, which is incorporated by reference. Purification methods for AAV8, WO2017/100676, filed Dec. 9, 2016, and rh10, WO2017/100704, filed Dec. 9, 2016, entitled “Scalable Purification Method for AAVrh10”, also filed Dec. 11, 2015, and for AAV1, WO2017/100674, filed Dec. 9, 2016 for “Scalable Purification Method for AAV1”, filed Dec. 11, 2015, are all incorporated by reference herein. Other suitable methods may be selected.

To calculate empty and full particle content, VP3 band volumes for a selected sample (e.g., in examples herein an iodixanol gradient-purified preparation where # of genome copies (GC)=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In certain embodiments, the manufacturing process for rAAV as described herein involves method as described in U.S. Provisional Patent Application No. 63/371,597, filed Aug. 16, 2022, and U.S. Provisional Patent Application No. 63/371,592, filed Aug. 16, 2022, which are incorporated herein by reference in its entirety.

In brief, the method for separating rAAVhu68 (or AAVhu95 or AAVhu96) particles having packaged genomic sequences from genome-deficient AAVhu68 (or AAVhu95 or AAVhu96) intermediates involves subjecting a suspension comprising recombinant AAVhu68 (or AVhu95 or AAVhu96) viral particles and AAVhu68 (or AVhu95 or AAVhu96) capsid intermediates to fast performance liquid chromatography, wherein the AAVhu68 (or AVhu95 or AAVhu96) viral particles and AAVhu68 (or AVhu95 or AAVhu96) intermediates are bound to a strong anion exchange resin equilibrated at a pH of about 10.2, and subjected to a salt gradient while monitoring eluate for ultraviolet absorbance at about 260 nanometers (nm) and about 280 nm. Although less optimal for rAAVhu68 (or AVhu95 or AAVhu96), the pH may be in the range of about 10 to 10.4. In this method, the AAV full capsids are collected from a fraction which is eluted when the ratio of A260/A280 reaches an inflection point. In one example, for the Affinity Chromatography step, the diafiltered product may be applied to an affinity resin (Life Technologies) that efficiently captures the AAV serotype. Under these ionic conditions, a significant percentage of residual cellular DNA and proteins flow through the column, while AAV particles are efficiently captured.

The rAAV.hLamin A (e.g., rAAV.CMVe.chTNTp.Lamin.RBG) is suspended in a suitable physiologically compatible composition (e.g., a buffered saline). This composition may be frozen for storage, later thawed and optionally diluted with a suitable diluent. Alternatively, the vector may be prepared as a composition which is suitable for delivery to a patient without proceeding through the freezing and thawing steps.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, the terms “rAAV” and “artificial AAV” used interchangeably, mean, without limitation, a AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine—glycine pairs.

### Pharmaceutical Composition

In one aspect, provided herein is a pharmaceutical composition comprising a vector as described herein in a formulation buffer. In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

Provided herein also is a composition comprising an rAAV or a vector as described herein and an aqueous suspension media. In certain embodiments, the suspension is formulated for intravenous delivery, intrathecal administration, or intracerebroventricular administration. In one aspect, the compositions contain at least one rAAV stock and an optional carrier, excipient and/or preservative.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. In one embodiment, the surfactant may be present in an amount up to about 0.0005% to about 0.001% (based on weight ratio, w/w %) of the suspension. In another embodiment, the surfactant may be present in an amount up to about 0.0005% to about 0.001% (based on volume ratio, v/v %) of the suspension. In yet another embodiment, the surfactant may be present in an amount up to about 0.0005% to about 0.001% of the suspension, wherein n % indicates n gram per 100 mL of the suspension.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrol® F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In certain embodiments, the composition containing the rAAV.hLamin A is delivered at a pH in the range of 6.8 to 8, or 7.2 to 7.8, or 7.5 to 8. In certain embodiments, the composition containing the rAAV.hLamin A is delivered intravenously at a pH of about 6.5 to about 7.5 may be desired. In certain embodiments, the composition containing the rAAV.hLamin A is delivered intravenously at a pH of about 6.8 to about 7.2 may be desired. However, other pHs within the broadest ranges and these subranges may be selected for other route of delivery.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. In one embodiment, the buffer is PBS.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, or for delivery by another route and/or device.

In one embodiment, a therapeutically effective amount of said vector is included in the pharmaceutical composition. The selection of the carrier is not a limitation of the present invention. As used herein, a “therapeutically effective amount” refers to the amount of the composition comprising the nucleic acid sequence encoding hLamin A (or an rAAV or a vector thereof) which delivers and expresses in the target cells an amount of protein sufficient to achieve efficacy. In one embodiment, the dosage of the vector is about 1×10⁹ GC/kg mass to about 1×10¹⁴ GC/kg, including all integers or fractional amounts within the range and the endpoints. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

It should be understood that the compositions in the pharmaceutical composition described herein are intended to be applied to other compositions, regiments, aspects, embodiments and methods described across the Specification.

### Methods and Uses

In one aspect, a method is provided herein is a method of treating a human subject diagnosed with idiopathic dilated cardiomyopathy (DCM) or a disease associated with a mutation in the LMNA gene. Further provided herein are use of an rAAV in the manufacture (preparing) of a medicament for the treatment a human subject diagnosed with idiopathic dilated cardiomyopathy (DCM) or a disease associated with a mutation in the LMNA gene.

The method comprises administering to a subject a suspension of a vector or an rAAV as described herein. In one embodiment, the method comprises administering to a subject a suspension of a rAAV as described herein in a formulation buffer at a dose of about 1×10′ genome copies (GC)/kg to about 1×10¹⁴ GC/kg. In a further embodiment, the rAAV is formulated at 3×10¹³ GC/kg.

In certain embodiments, the method of treatment of idiopathic DCM or a disease associated with a mutation in LMNA gene further comprises monitoring hLaminA expression and percent cardiomyocyte transduction using endomyocardial biopsy.

The methods and compositions described herein may be used for treatment of any of the stages of idiopathic dilated cardiomyopathy (DCM) or a disease associated with a mutation in a Lamin A (LMNA) gene. In certain embodiments, the patient is an infant, a toddler, or the patient is from 3 years to 6 years of age, from 3 years to 12 years of age, from 3 years to 18 years of age, from 3 years to 20 years of age. In certain embodiments, patients are older than 18 years of age. In certain embodiments, the patient is about 20 to 60. In certain embodiments, the patient is about 40 to 50. In certain embodiment, patients are older than 60 years of age.

In certain embodiments, the methods and compositions may be used for treatment of adult-onset form of dilated cardiomyopathy with conduction defects. In certain embodiments, the methods and compositions may be used for treatment of LMNA cardiomyopathy caused by loss-of-function mutation in the LMNA gene. In certain embodiments, the methods and compositions may be used for treatment of LMNA cardiomyopathy which is autosomal dominant inheritance. In certain embodiments, the methods and compositions may be used for treatment of early onset phenotype disease (e.g., idiopathic DCM) associated with nonsense mutations in LMNA gene. In certain embodiments, the methods and compositions may be used for treatment of LMNA cardiomyopathy (e.g., idiopathic DCM) associated with missense and truncation in LMNA gene. In certain embodiments, the methods and compositions may be used for treatment of LMNA cardiomyopathy (e.g., idiopathic DCM) associated with Q15X mutation in LMNA gene. In certain embodiments, the methods and compositions may be used for treatment of LMNA cardiomyopathy (e.g., idiopathic DCM) associated with N195K mutation in LMNA gene.

Additionally, other diseases may be associated with a mutation in an LMNA gene including muscular dystrophy, neuropathy, lipodystrophy, segmental progeroid. For example, diseases associated with a mutation in an LMNA gene include Emery-Dreifuss Muscular dystrophy (EDMD), Malouf syndrome (MLF), Congenital Muscular dystrophy (MDC), Limb-Gardle Muscular dystrophy type 1B (LGMD1B), Charcot-Marie-Tooth disease type 2B1 (CMT2B1), axonal neuropathy, familial partial lipodystrophy type 2 (FPLD2), Mandibuloacral dysplasia lipodystrophy (MAD), Mandibuloacral Dysplasia type A (MADA), Atypical Werner syndrome (AWS), premature aging syndrome (progeria) and Hutchinson-Gilford progeria syndrome (HGPS).

Symptoms of idiopathic dilated cardiomyopathy (DCM) or a disease associated with a mutation in a Lamin A (LMNA) gene include atrioventricular (AV) conduction block, atrial fibrillation, atrial arrhythmia including atrial flutter and atrial tachycardia, ventricular arrhythmias including sustained ventricular tachycardias and ventricular fibrillation (VF). In certain embodiments, the methods and compositions described herein are used to ameliorate or improve one or more symptoms of idiopathic dilated cardiomyopathy (DCM) or a disease associated with a mutation in a Lamin A (LMNA) gene including atrioventricular (AV) conduction block, atrial fibrillation, atrial arrhythmia, including atrial flutter and atrial tachycardia, ventricular arrhythmias including sustained ventricular tachycardias and ventricular fibrillation (VF) dilated cardiomyopathy, and/or heart failure. In certain embodiments, the methods and compositions described herein may be used to ameliorate one or more symptoms of idiopathic dilated cardiomyopathy (DCM) or a disease associated with a mutation in a Lamin A (LMNA) gene including increased average life span, and/or reduction in progression towards heart failure.

In certain embodiments, co-therapies or co-treatments may be utilized, which comprise co-administration with another active agent. In certain embodiments, the co-therapy may further comprise administration of beta blockers, angiotensin-converting enzyme (ACE) inhibitors, diuretics. A diuretic agent used may be acetazolamine (Diamox) or other suitable diuretics. In some embodiments, the diuretic agent is administered at the time of gene therapy administration. In some embodiments, the diuretic agent is administered prior to gene therapy administration. In some, embodiments the diuretic agent is administered where the volume of injection is 3 mL. In certain embodiments, the co-treatment may further comprise implantable cardioverter defibrillators (ICD), pacemakers (PM) and/or cardiac resynchronization therapy (CRT).

Optionally, an immunosuppressive co-therapy may be used in a subject in need. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-0, IFN-γ, an opioid, or TNF-α (tumor necrosis factor-alpha) binding agent. In certain embodiments, the immunosuppressive therapy may be started 0, 1, 2, 3, 4, 5, 6, 7, or more days prior to or after the gene therapy administration. Such immunosuppressive therapy may involve administration of one, two or more drugs (e.g., glucocorticoids, prednelisone, micophenolate mofetil (MMF) and/or sirolimus (i.e., rapamycin)). Such immunosuppressive drugs may be administrated to a subject in need once, twice or for more times at the same dose or an adjusted dose. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

In one embodiment, the rAAV as described herein is administrated once to the subject in need. In another embodiment, the rAAV is administrated more than once to the subject in need.

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human patient. In certain embodiment, the subject of these methods and compositions is diagnosed with idiopathic dilated cardiomyopathy (DCM) or a disease associated with a mutation in a Lamin A (LMNA) gene and/or with symptoms of idiopathic dilated cardiomyopathy (DCM) or a disease associated with a mutation in a Lamin A (LMNA) gene.

It should be understood that the compositions in the method described herein are intended to be applied to other compositions, regiments, aspects, embodiments and methods described across the Specification.

### Kit

In certain embodiments, a kit is provided which includes a concentrated vector suspended in a formulation (optionally frozen), optional dilution buffer, and devices and components required for intravenous administration. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in kit described herein are intended to be applied to other compositions, regiments, aspects, embodiments and methods described across the Specification.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

As described above, the terms “increase” “decrease” “reduce” “ameliorate” “improve” “delay” or any grammatical variation thereof, or any similar terms indication a change, means a variation of about 5 fold, about 2 fold, about 1 fold, about 90%, about 80%, about 70%, about 60%, about 50%, about 40%, about 30%, about 20%, about 10%, about 5% compared to the corresponding reference (e.g., untreated control or a subject in normal condition without idiopathic dilated cardiomyopathy (DCM) or a disease associated with a mutation in a Lamin A (LMNA) gene), unless otherwise specified.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, the term “administration” or any grammatical variations thereof refers to delivery of composition described herein to a subject.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be included and described using “consisting of” or “consisting essentially of” language. As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an” refers to one or more. As such, the terms “a” (or “an”), “one or more,” and “at least one” are used interchangeably herein.

As used herein, the term “about” or “˜” refers to a variant of 10% from the reference integer and values therebetween, unless otherwise specified. For example, “about” 500 μM includes ±50 (i.e., 450-550, which includes the integers therebetween). For other values, particularly when reference is to a percentage (e.g., 90% of taste), the term “about” is inclusive of all values within the range including both the integer and fractions.

As described above, the term “about” when used to modify a numerical value means a variation of 10%, (±10%, e.g., ±1, +2, +3, +4, +5, +6, +7, +8, +9, +10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are provided to illustrate certain aspects of the claimed invention. The invention is not limited to these examples.

There is an extremely high and unmet need in the adult-onset form of dilated cardiomyopathy (DCM) with conduction defects, wherein complete penetrance with high rate of transplant and death is observed. In some cases, DCM is caused by loss-of-function mutations in the Lamin A (LMNA) gene, which makes it amenable to gene replacement therapy. DCM has an autosomal dominant inheritance pattern, therefore the presence of the residual normal protein reduces risk of immune response to transgene being delivered in cases of gene replacement therapy. Since there are approximately 40,000 symptomatic LMNA-related cardiomyopathy (or DCM) patients in the US, there is a large addressable patient population.

Missense and truncation mutations are common in DCM families. Nonsense mutations and observed in more severe and/or earlier onset phenotype (Hasselberg et al. European Heart Journal (2018) 39, 853-860; Suguru Nishiuchi. Circulation: Cardiovascular Genetics. Gene-Based Risk Stratification for Cardiac Disorders in LMNA Mutation Carriers, Volume: 10, Issue: 6). In some cases of DCM, in carriers of very early truncation mutations of LMNA gene demonstrates lack of dominant negative function (e.g., Q15X). There are LMNA knockout and LMNA mutations mouse models available to address the function of Lamin A. For example, mice carrying N195K DCM mutation has a less severe phenotype than KO mice.

Here, we have developed an AAV vector expressing mature human Lamin A (hLamin A) from a cardiac selective promoter that rescues the lethal phenotype of LMNA knockout mice.

### Example 1. Production of rAAV Comprising Lamin A

In the studies herein, an engineered mature human LaminA (also referred to as hLaminA, hLamin A, huLaminA, huLamin A, hLMNA) sequence and rAAV comprising mature hLamin A were generated and comparative studies were performed. In some cases, rAAV comprising GFP gene specified promoters were generated and used to evaluate promoter-driven cardiac transgene expression in mice.

The rAAV are generated using triple transfection techniques, utilizing (1) a cis plasmid encoding AAV2 rep proteins and the AAVhu68 VP1 cap gene, (2) a cis plasmid comprising adenovirus helper genes not provided by the packaging cell line which expresses adenovirus Ela, and (3) a trans plasmid containing the vector genome for packaging in the AAV capsid. See, e.g., US 2020/0056159. The trans plasmid is designed to contain either the vector genome comprising huLamin A.

The vector genome contains an AAV 5′ inverted terminal repeat (ITR) and an AAV 3′ ITR at the extreme 5′ and 3′ end, respectively. The ITRs flank the sequences of the expression cassette packaged into the AAV capsid which have sequences encoding a mature Lamin A. The expression cassette further comprises regulatory sequences operably linked to the fusion protein coding sequences, the regulatory control sequence of which includes a hybrid cardiac promoter comprising a CMV IE enhancer, a spacer sequence, and a chicken cardiac troponin T (chTnT), wherein the expression cassette further includes a rabbit beta-globin (RBG) polyA. SEQ ID NO: 1 refers to expression cassette of CMV-IE.chTnT.mature_hLaminA.rBG. SEQ ID NO: 2 refers to vector genome of rAAV.CMV-IE.chTnT.mature_hLaminA.rBG, also referred to below as rAAV.hLaminA.

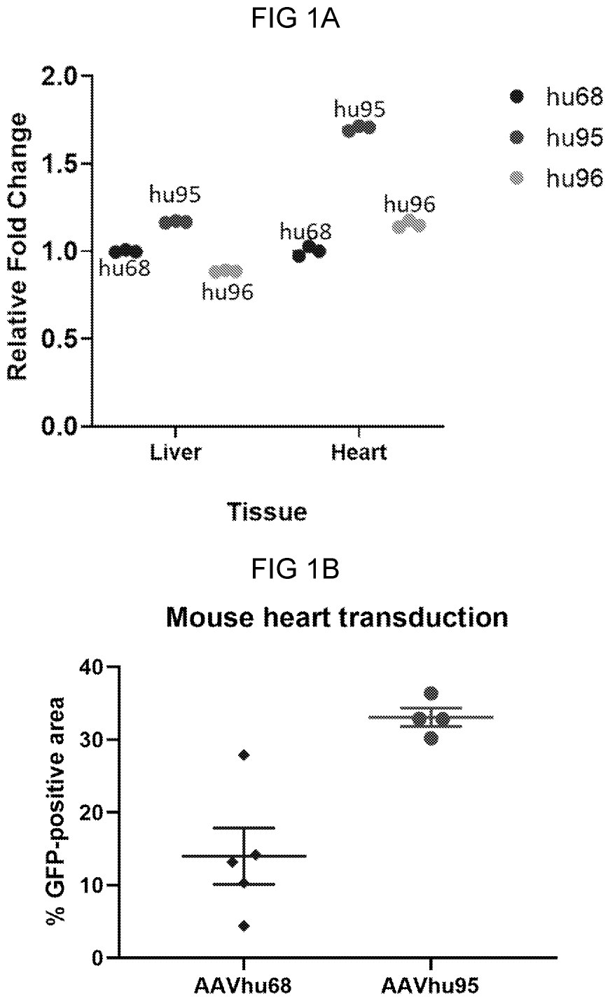

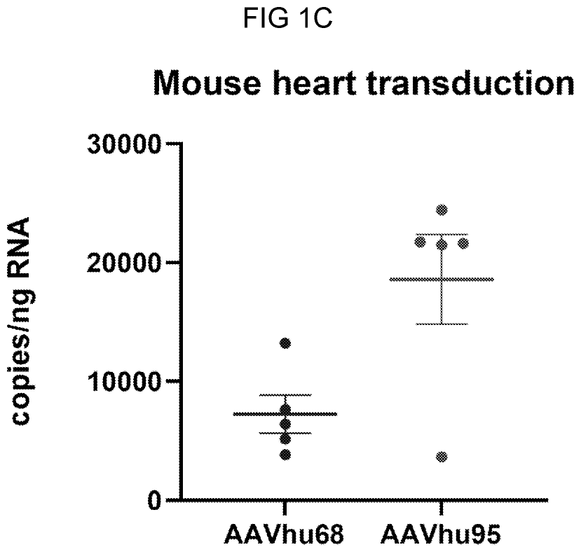

Additionally, we examined efficacy of using various AAV capsid to achieve cardiac specific AAV transduction. We examined AAVhu95 capsid and AAVhu96 capsid. AAVhu95 capsid and AAVhu96 are Clade F AAV capsids which were isolated from human tissue. AAVhu95 capsid and AAVhu96 capsid are closely related to AAVhu68 capsid, and has similar production yields. Furthermore, upon examination, AAVhu95 capsid showed moderate improvement in cardiac transduction relative to AAVhu68 capsid. Briefly, in this study, a single NHP was intravenously administered a combination of 3 vectors (AAVhu68, AAVhu95, and AAVhu96) each expressing a unique barcode (3×10¹³ GC/vector). FIG. 1A shows relative levels of gene transfer to NHP heart, plotted as fold change in RNA sequencing reads (prevalence of RNA reads in tissue relative to vector concentration administered) relative to AAVhu68. Similarly, mice were treated with 10¹² GC of the indicated vector (AAVhu95 or AAVhu68) expressing GFP. Animals were sacrificed 14 days after vector administration and vector RNA copies and GFP fluorescence-positive area were quantified from heart samples. FIG. 1B shows levels of transduction in mouse heart following administration with AAVhu68 or AAVhu95 comprising gene encoding for Green Fluorescent Protein, plotted as percent of GFP-positive area. FIG. 1C shows levels of transduction in mouse heart following administration with AAVhu68 or AAVhu95 comprising gene encoding for Green Fluorescent Protein, plotted as number of copies/ng RNA.

### Example 2. Pilot Study Examining Efficacy of rAAV Comprising Lamin a in Lamin a Knockout Mouse Model

In this study, we used an AAVhu95 capsid and an AAVhu68 capsid (in initial mouse study), encoding a mature Lamin A under regulatory control of hybrid cardiac promoter comprising a CMV IE enhancer, a spacer sequence, and a cardiac selective promoter (cardiac Troponin T or TnT). The use of engineered sequence encoding mature Lamin A avoids potential toxicity of un-cleaved farnesylated prelamin. Additionally, famesylated portion of the Lamin A (not included in the mature Lamin A) appears unnecessary for function. Furthermore, Lamin C only mice are phenotypically normal.

Briefly, Lamin A knock out (KO) and wild type (WT) newborn littermates (N-7-10 per group) were injected intravenously (via temporal vein injection) with either vehicle (PBS) or AAVhu68.huLaminA at a dose of 5×10¹⁰ GC (5e10 GC, about 3×10¹³ GC/kg (3e13 GC/kg)) at post natal day 0 (approximate dose assuming Ig weight 5e13 GC/kg). Mice survival was examined, and body weights were measured throughout study, at the indicated days. Survival and cardiac echographic parameters were recorded for a maximal duration of 12 weeks. Treatment shows survival rescue; cardiac parameters are not clearly improved by echo (severe model likely defects prior to treatment at birth). In vivo spectral and tissue doppler Interval measurements were performed along with two-dimensional B-Mode and M-Mode transthoracic echocardiography on all mice at P42 using Vevo 3100 Ultrasound Imaging System (FUJIFILM Visual Sonics) and a 40 Mhz transducer. Mice were anesthetized via 2% isoflurane in an induction chamber for 3 minutes, then moved to an animal monitor platform with ECG capability and thermoregulation controlled by feedback from a rectal thermometer. There, the mice were fitted with a nose cone (1.5% isoflurane to maintain plane of anesthesia). Nair was used to remove hair before imaging. The researchers performing echo acquisition and analysis were blind to study groups. Analysis was performed using Vevo LAB software (FUJIFILM VisualSonics).

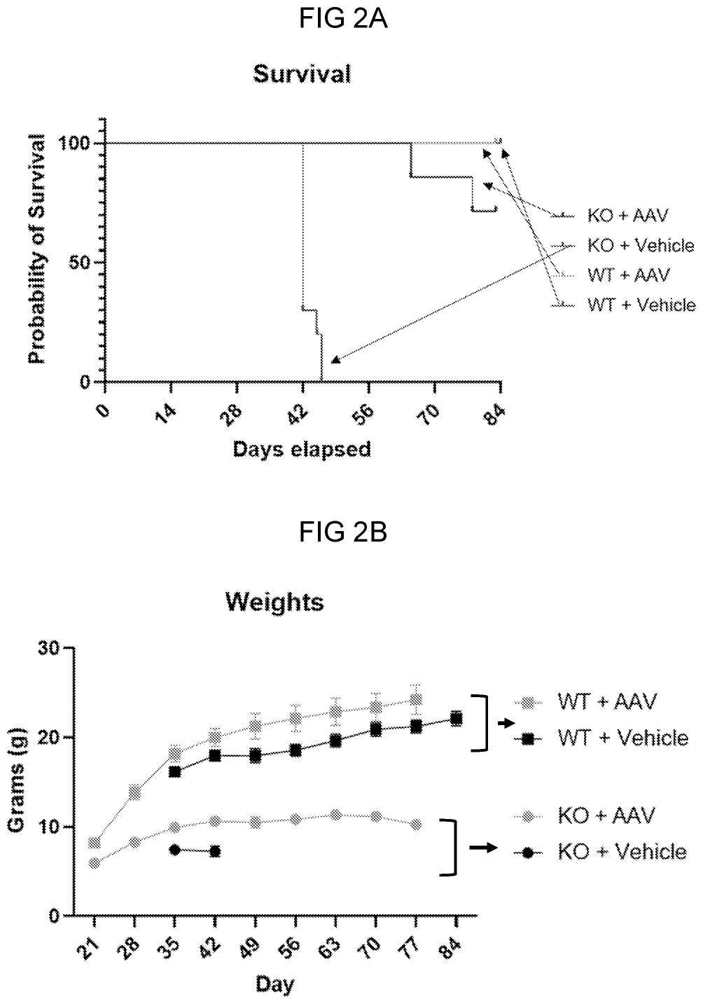

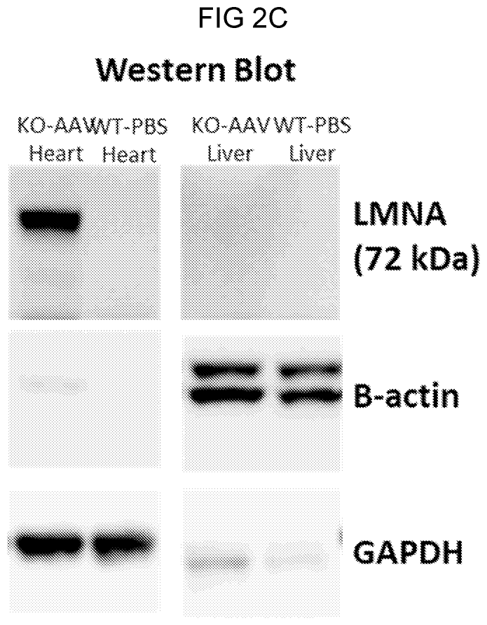

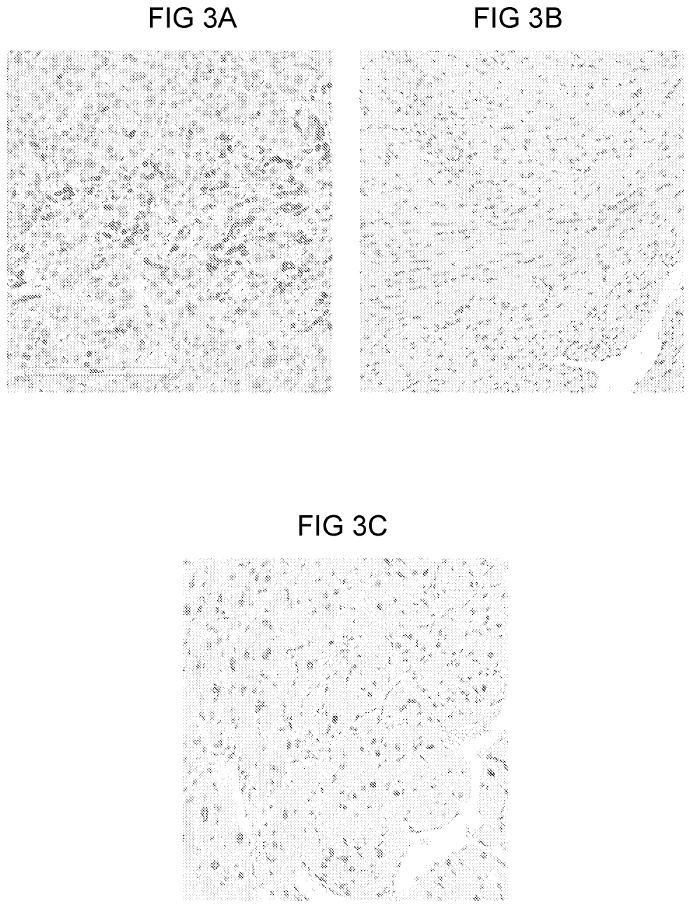

FIG. 2A shows a Kaplan-Meier survival plot of Lamin knockout (KO) and Wild type (WT) mice administered at newborn stage with either a vehicle control or AAVhu68.hLaminA intravenously at a dose of 5×10¹⁰ GC (approximately 3×10¹³ GC/kg). FIG. 2B shows measured body weights of Lamin knockout (KO) and Wild type (WT) mice administered at newborn stage with either a vehicle control or AAVhu68.hLaminA intravenously at a dose of 5×10¹⁰ GC (approximately 3×10¹³ GC/kg). FIG. 2C shows a representative western blot confirming expression of LaminA in heart and lack of expression of Lamin A in liver following administration of AAVhu68.hLaminA in knock-out mice. These results confirm Lamin A expression in heart tissue, and show increased survival in knock-out mice with AAV-LMNA treatment. Upon necropsy, liver and heart tissues were collected and analyzed with immunohistochemical staining with anti-human lamin antibody to examine expression of mature huLamin A (FIGS. 3A-3C).

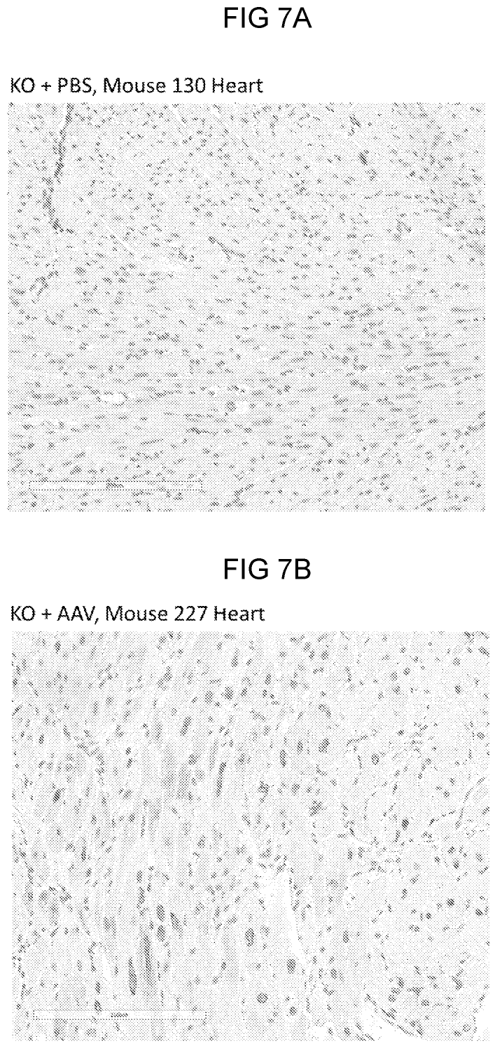

FIG. 3A shows a representative microscopy image from immunohistochemical (IHC) analysis of staining with anti-human lamin of FRG mouse liver tissue, following administration at newborn stage with AAVhu68.hLaminA intravenously at a dose of 5×10¹⁰ GC (approximately 3×10¹³ GC/kg). FIG. 3B shows a representative microscopy image from immunohistochemical (IHC) analysis of staining with anti-human lamin of mouse heart tissue, following administration at newborn stage with vehicle control. FIG. 3C shows a representative microscopy image from immunohistochemical (IHC) analysis of staining with anti-human lamin of mouse heart tissue, following administration at newborn stage with AAVhu68.hLaminA intravenously at a dose of 5×10¹⁰ GC (approximately 3×10¹³ GC/kg). FIG. 7A shows a representative image of histology analysis of heart tissue in knock out mice following administration of PBS in knock-out mice. FIG. 7B shows a representative image of histology analysis of heart tissue in knock out mice following administration of AAV-LMNA in knock-out mice, confirming expression of LMNA in ventricular cardiac cells.

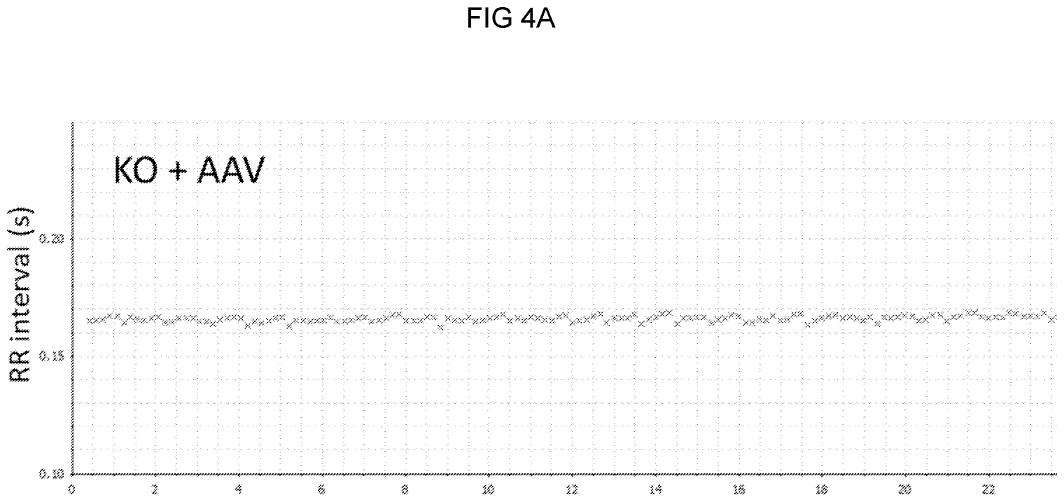

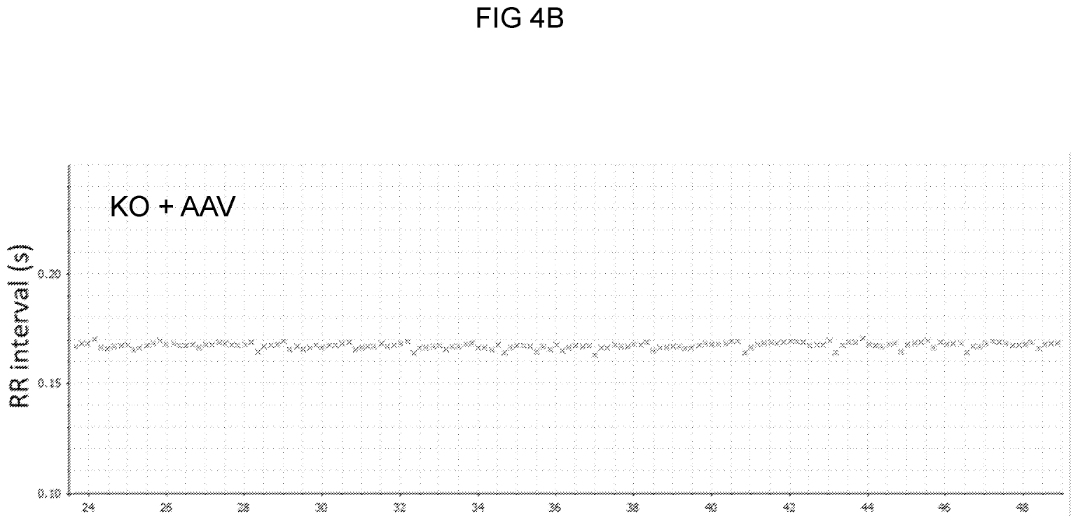

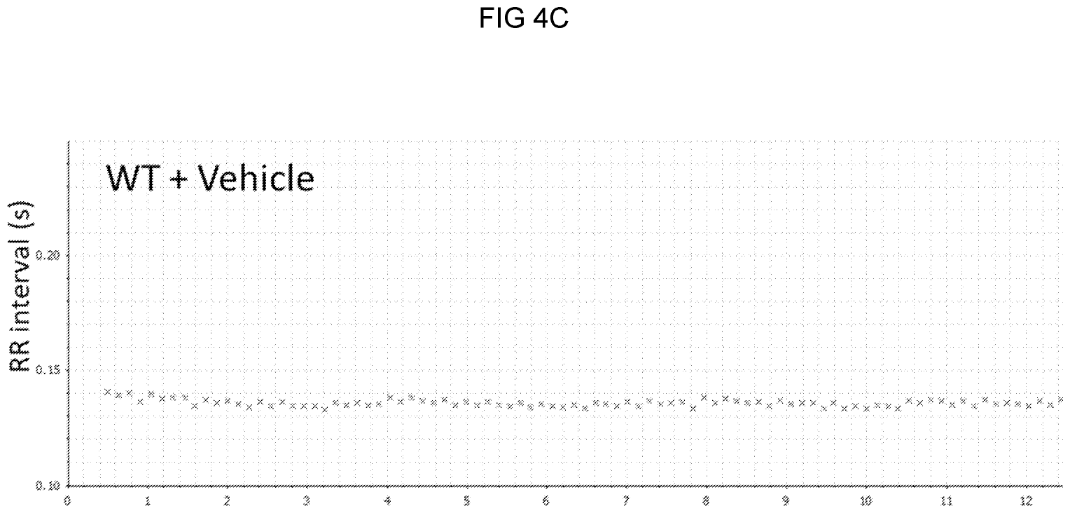

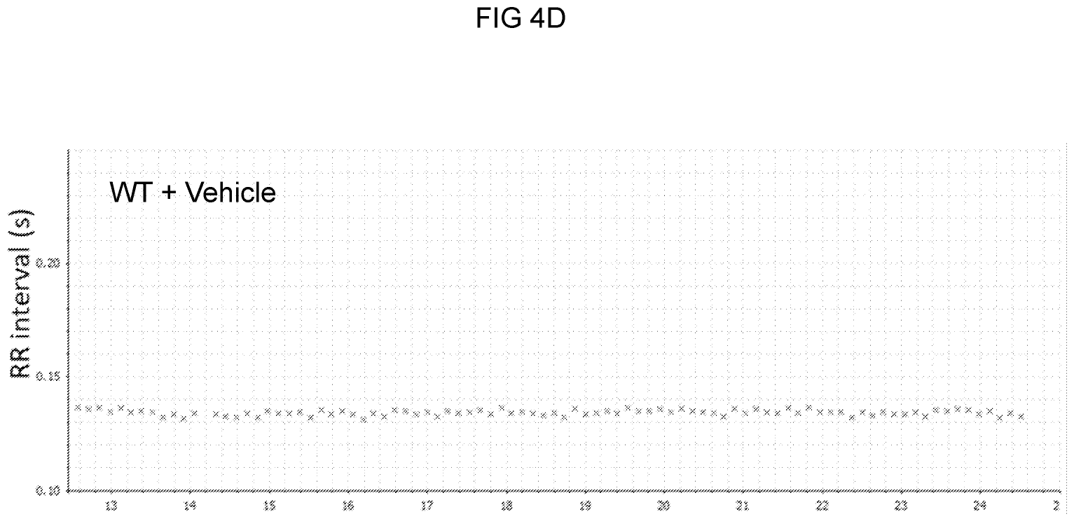

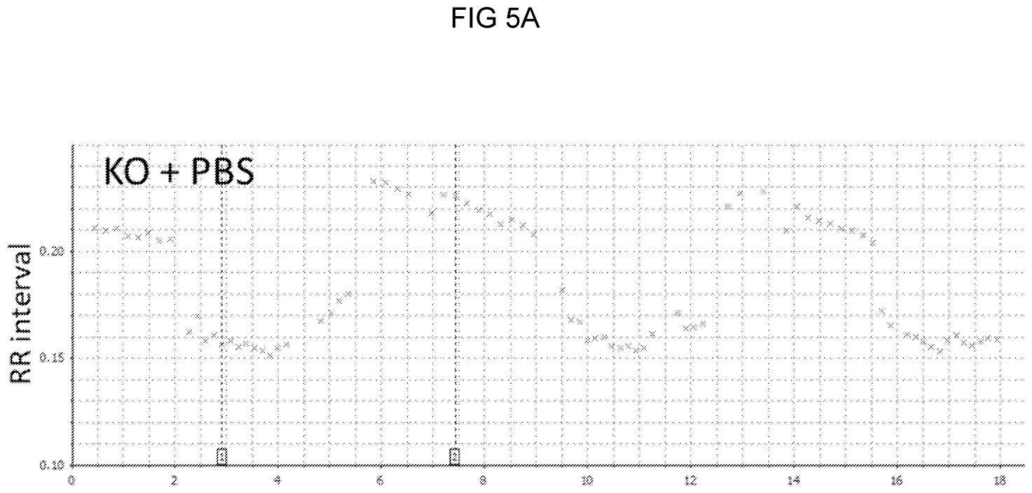

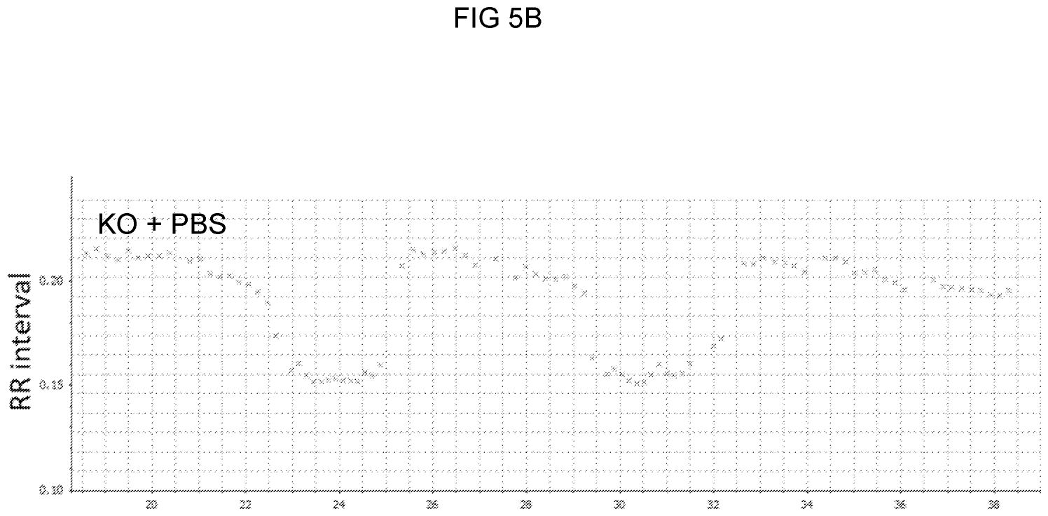

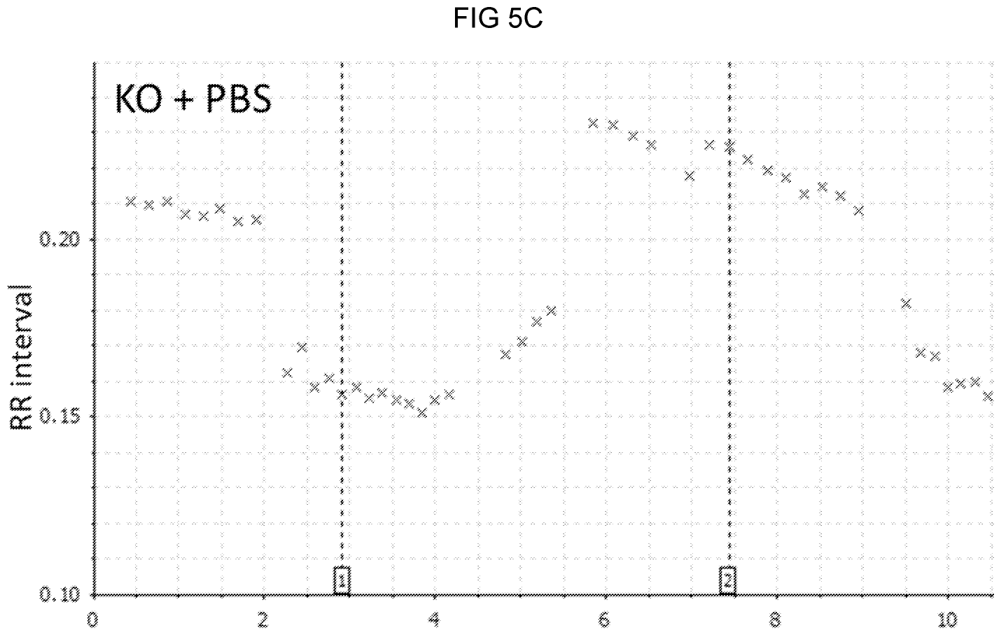

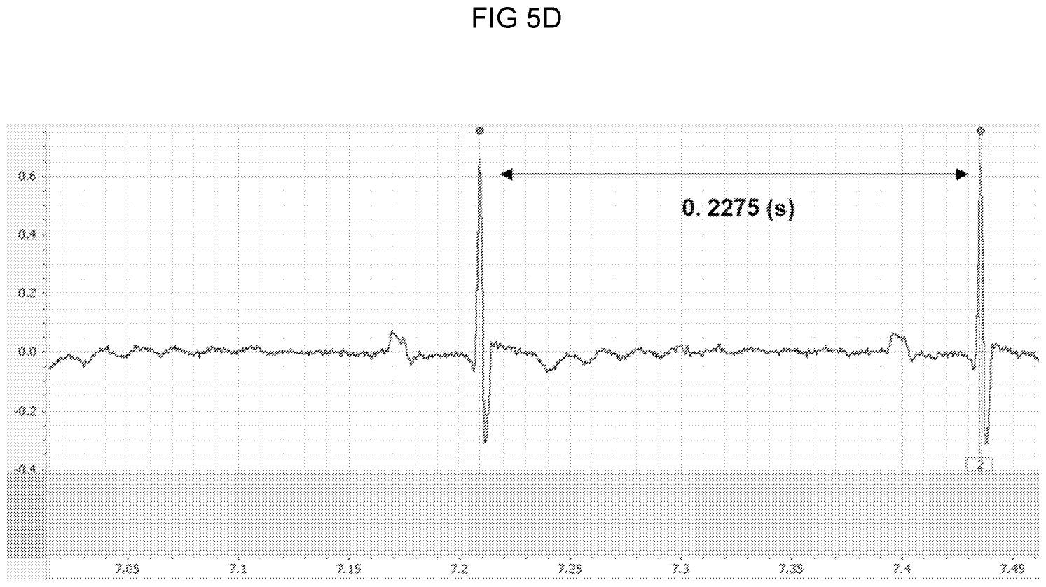

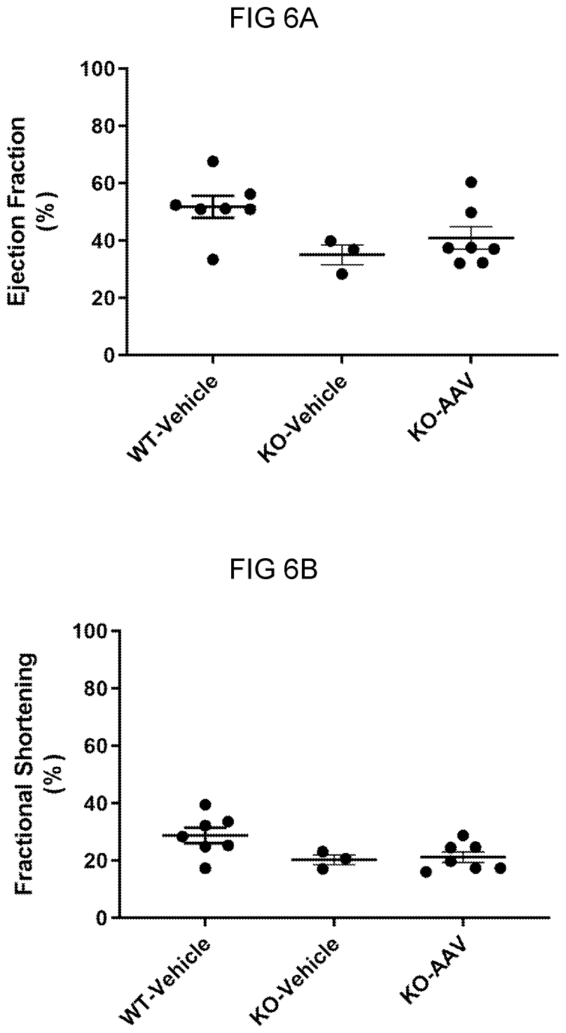

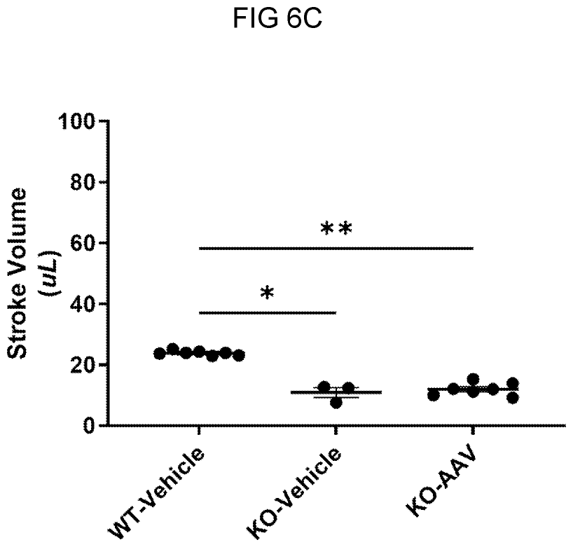

Next, we examined the efficacy of treatment with rAAV.hLaminA on cardiac rhythm in Lamin A KO mice. Electrocardiogram and echocardiogram were performed in accordance to the well-established and published protocols in literature. FIG. 4A shows a representative cardiogram analysis showing RR Interval (s) over time, in Lamin A KO mice administered with AAV (0-23). FIG. 4B shows a representative cardiogram analysis showing RR Interval (s) over time, in Lamin A KO mice administered with AAV (24-48). FIG. 4C shows a representative cardiogram analysis showing RR Interval (s) over time in WT mice administered with vehicle (PBS) (0-12). FIG. 4D shows a representative cardiogram analysis showing RR Interval (s) over time in WT mice administered with vehicle (PBS) (13-26). In comparison, we observed bradycardia in vehicle (PBS)-treated Lamin A KO mice (FIGS. 5A to 5D). FIG. 5A shows a representative cardiogram analysis showing RR Interval (s) over time in Lamin A KO mice administered with vehicle (PBS) (0-18). FIG. 5B shows a representative cardiogram analysis showing RR Interval (s) over time in Lamin A KO mice administered with vehicle (PBS) (19-38). FIG. 5C shows a representative cardiogram analysis showing RR Interval (s) over time in Lamin A KO mice administered with vehicle (PBS) (zoomed in 5A, time 0-10). FIG. 5D shows a representative cardiogram analysis showing RR Interval (s) over time in Lamin A KO mice administered with vehicle (PBS) (zoomed in 5B, time 7.00-7.45). FIG. 6A shows results of the echocardiogram in wild-type (WT) and knock out (KO) mice administered with vehicle (PBS) or AAV, plotted as ejection fraction (%) (one-way Anova non-parametric Kruskal-Wallis with Dunn's multiple comparison test: P<0.05, **P<0.01, ***P<0.01, ****P<0.0001). FIG. 6B shows results of the echocardiogram in wild-type (WT) and knock out (KO) mice administered with vehicle (PBS) or AAV, plotted fractional shortening (%) (one-way Anova non-parametric Kruskal-Wallis with Dunn's multiple comparison test: P<0.05, **P<0.01, ***P<0.01, ****P<0.0001). FIG. 6C shows results of the echocardiogram in wild-type (WT) and knock out (KO) mice administered with vehicle (PBS) or AAV, plotted as stroke volume (μL) (one-way Anova non-parametric Kruskal-Wallis with Dunn's multiple comparison test: P<0.05, **P<0.01, ***P<0.01, ****P<0.0001).

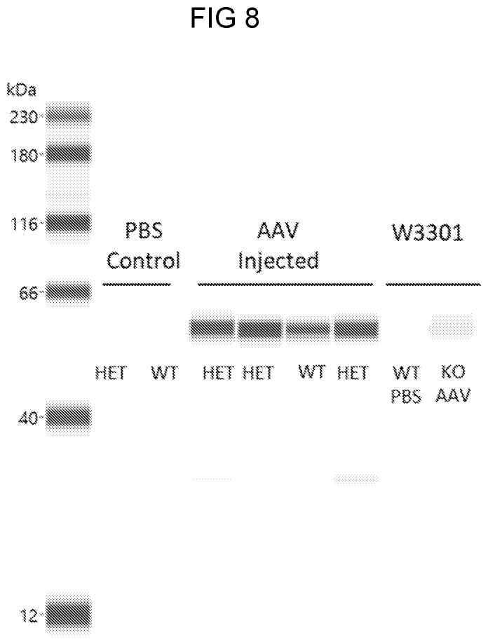

Additionally, we examined efficacy of AAVhu95.CMVe.chTNTp.LaminA.rBG (AAVhu95-LMNA) in mice. Briefly, Mice treated with AAVhu95-LMNA, and expression was examined with western blot at day 5. FIG. 8 shows a representative western blot analysis for LMNA expression in mice administered with AAVhu95-LMNA. The antibody used in the western blob analysis showed low affinity for endogenous mouse LMNA.

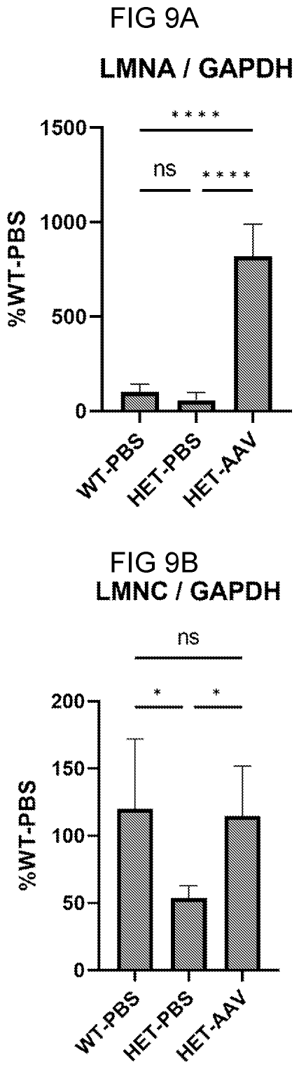

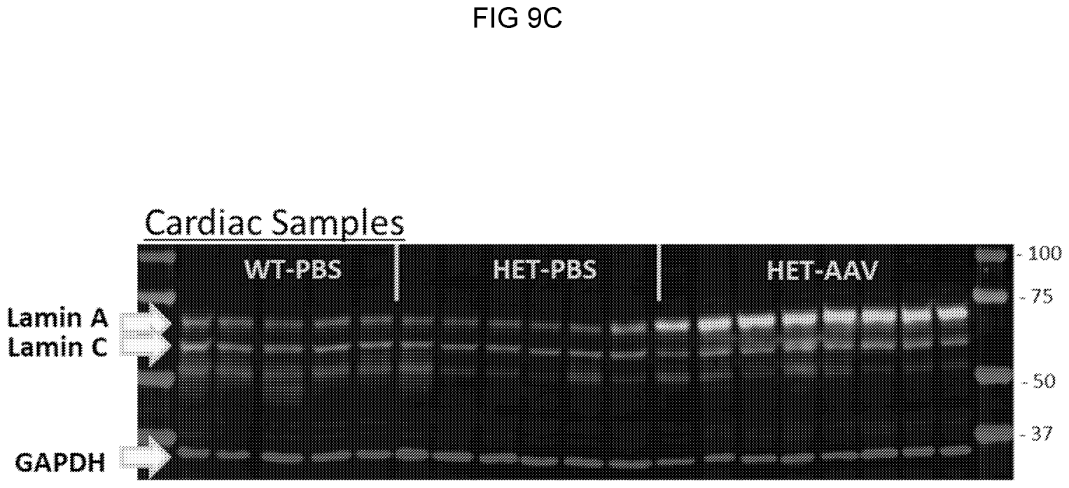

Additionally, we examined efficacy of AAVhu95-LMNA in heterozygous mice. FIG. 9A shows results of the LMNA telemetry study plotted as percent of WT-PBS of LMNA expression in wild-type and heterozygous knockout mice following administration with either PBS (control) or AAV-LMNA. FIG. 9B shows results of the LMNA telemetry study plotted as percent of WT-PBS of LMNC expression in wild-type and heterozygous knockout mice following administration with either PBS (control) or AAV-LMNA. FIG. 9C shows a representative western blot analysis of cardiac samples for Lamin A and Lamin C expression in mice (wild type and heterozygous knock-out mice) administered with AAVhu95-LMNA. These results show that Lamin A expression is significantly increased in AAVhu95-LMNA-treared mice hearts at day 120.

In summary, in knock-out mouse phenotype model, we observed low survival rate past 6 weeks, several mice decreased in body weight, we also observed a trend toward decreased fractional shortening (FS) and ejection fraction (EF) and decreased stroke volume (SV) in echocardiogram, also we observed abnormally shaped nuclei, and suspected arrhythmia as a cause of cardiac arrest. In comparison, for AAV-treated mice, we observed increased survival of knock-out mice, confirmed increased LMNA expression in treated heterozygous knock-out mice.

### Example 3. Safety and Expression Study of rAAV.hLaminA in Mice and Nonhuman Primates (NHPs)

In this study, we perform a pharmacology study with telemetry to evaluate arrhythmias as a cause of death. We further perform additional echocardiogram (ECG) and echo studies in aged mice heterozygous for laminopathy.

Next, we perform a safety and expression study in non-human primates. In this study, AAVhu95 expressing human lamin A are used (e.g., AAVhu95.CMV-IE.chTnT.hLaminA.rBG or AAVhu95.hLaminA or AAVhu95-LMNA). Briefly, NHPs (N=2) are administered intravenously with AAVhu95.hLaminA at a dose of 3×10¹³ GC/kg via 10 mL-infusion at 1 mL/min infusion rate. The study duration is 60 days (2 months), during which serial cage side observations, vitals, physical exam are performed throughout, and samples are taken for evaluation of CBC, serum chemistry, troponin I levels. Additionally, echo and ECG are performed at day 0 (i.e., baseline, before administration), 1 month and 2 months of the study duration. At the end of the duration of the study, necropsy is performed and tissues are collected for histopathology, and in situ hybridization analysis (ISH) for transgene expression in heart.

Expression cannot be done by protein IHC due to cross reactivity human/NHP LaminA. ISH was done but showed low expression compared to what was expected at this dose level. It is inconclusive if this is because hu95 performs less well in NHP compared to mouse or compared to the benchmark hu68.

Heterozygous mice do not have a lethal phenotype. This study was conducted to see if a phenotype could be seen by ECG and eventually rescued by treatment. Six months old HET mice received, AAVhu95M199.CMVe.chTNTp.laminA.rBG IV (tail vein) at a dose of 3×10¹³ GC/kg via tail vein injection or PBS as controls; WT PBS were used as controls. Regular ECG recordings were performed on these mice via implanted telemeters. Tissue was harvested at ˜D120 and used for Western Blot. No conclusive results from telemetry because of technical issues (electrodes did not stay in place) and because WT controls also have abnormal reading due to age. This study confirmed good expression on D120 suggesting the lack of expression in NHP was not due to the capsid unless hu95 behaves differently in mouse (good cardiac tropism) versus NHP.

### REFERENCES

1. Hershberger, R., Hedges, D. & Morales, A. Dilated cardiomyopathy: the complexity of a diverse genetic architecture. Nat Rev Cardiol 10, 531-547 (2013).
2. Parks S B, Kushner J D, Nauman D, et al. Lamin A/C mutation analysis in a cohort of 324 unrelated patients with idiopathic or familial dilated cardiomyopathy. Am Heart J 2008; 156:161-9.
3. Hasselberg et al. European Heart Journal (2018) 39, 853-860.
4. Kang et al. BMB Reports 2018; 51:327-37.
5. Charron, P., et al., What Should Cardiologist know about Lamin Disease?, Arrhythmia & Electrophysiology Review 2012; 1(1):22-8.
6. Suguru Nishiuchi, Circulation: Cardiovascular Genetics. Gene-Based Risk Stratification for Cardiac Disorders in LMNA Mutation Carriers, Volume: 10, Issue: 6.
7. Hasselberg et al. European Heart Journal (2018) 39, 853-860.
8. Kumar, S. et al., Long-Term Arrhythmic and Nonarrhythmic Outcomes of Lamin A/C Mutation Carriers, J Am Coll Cardiol. 2016 November, 68 (21) 2299-2307.
9. Ollila L., et al. Clinical disease presentation and ECG characteristics of LMNA mutation carriers, Open Heart 2017; 4:e000474.
10. Rubinstein L. V., Gail M. H., Santner T. J., Planning the duration of a comparative clinical trial with loss to follow-up and a period of continued observation, (1981) J Chron Dis 8: 67-74.

All documents cited in this specification are incorporated herein by reference are incorporated by reference. U.S. Provisional Patent Application No. 63/293,680, filed Dec. 24, 2021, which is incorporated herein by reference in its entirety. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) comprising an adeno-associated virus (AAV) capsid and a vector genome packaged in the AAV capsid, wherein the vector genome comprises an AAV 5′ inverted terminal repeat (ITR), an expression cassette, and an AAV 3′ ITR, wherein the expression cassette comprises an engineered open reading frame (ORF) for mature human Lamin A (hLaminA) coding sequence which encodes for mature hLaminA lacking the preprotein carboxy (C) terminus tail, wherein the ORF is operably linked to regulatory control sequences which direct expression of the mature hLaminA protein in a cell, and wherein the regulatory control sequences comprise a promoter, optionally an enhancer, and a polyadenylation (polyA) sequence.

2. The rAAV according to claim 1, wherein the engineered ORF comprises coding sequence having the nucleic acid sequence of SEQ ID NO: 4 or a nucleic acid sequence at least 90% identical to SEQ ID NO: 4 which encodes mature hLaminA lacking the preprotein carboxy (C) terminus tail.

3. The rAAV according to claim 1, wherein the mature hLaminA has the amino acid sequence of SEQ ID NO: 5.

4. The rAAV according to claim 1, wherein the mature hLaminA coding sequence comprises nucleic acid sequence of SEQ ID NO: 4.

5. The rAAV according to claim 1, wherein the regulatory control sequences comprise a promoter which is a cardiac promoter.

6. The rAAV according to claim 5, wherein the cardiac promoter is cardiac troponin T (cTnT) promoter.

7. The rAAV according to claim 6, wherein the cTnT promoter is a chicken cTnT promoter.

8. The rAAV of claim 1, wherein the regulatory control sequences comprise a hybrid cardiac promoter comprising a cytomegalovirus immediate early (CMV IE) enhancer, a spacer sequence, and a chicken cTNT promoter.

9. The rAAV of claim 1, wherein the regulatory control sequences comprise polyA sequence which is a rabbit beta-globin (rBG) polyA sequence.

10. The rAAV of claim 1, wherein the regulatory control sequences further comprise a mutant WPRE element.

11. The rAAV of claim 1, wherein the expression cassette comprises a hybrid cardiac promoter comprising a CMV IE enhancer, a spacer sequence and a chicken cTnT promoter, wherein the hybrid promoter is operably linked to the hLaminA coding sequence of SEQ ID NO: 4, and the expression cassette further comprises a rBG poly A sequence.

12. The rAAV according to claim 11, wherein the expression cassette comprises a nucleic acid sequence of SEQ ID NO: 2 or a nucleic acid sequence at least 90% identical to SEQ ID NO: 2.

13. (canceled)

14. The rAAV of claim 1, wherein the AAV capsid is a Clade F AAV.

15. The rAAV of claim 1, wherein the AAV capsid is AAVhu68, AAVhu95, or AAVhu96.

16-20. (canceled)

21. A composition comprising a stock of rAAV of claim 1 and an aqueous suspension media.

22-28. (canceled)

29. A recombinant nucleic acid molecule comprising an expression cassette an engineered open reading frame (ORF) for mature human Lamin A (hLaminA), wherein the ORF has a mature hLaminA coding sequence, which is a nucleic acid sequence encoding a functional mature human hLaminA lacking the preprotein carboxy (C) terminus tail, wherein ORF is operably linked to regulatory control sequences, said expression cassette being flanked by a 5′ inverted terminal repeat (ITR) and a 3′ ITR, wherein the engineered hLaminA gene encodes a functional mature hLamin A lacking the preprotein carboxy (C) terminus tail, and wherein the hLaminA coding sequence comprises nucleic acid sequence of SEQ ID NO: 4.

30-31. (canceled)

32. A packaging host cell comprising a nucleic acid molecule of claim 31.

33-34. (canceled)

35. An rAAV production system useful for producing the rAAV of claim 1, wherein the production system comprises a cell culture comprising:
(a) a nucleic acid sequence encoding a AAV capsid protein;
(b) a vector genome; and
(c) sufficient AAV rep functions and helper functions to permit packaging of the vector genome into the AAV capsid.

36. (canceled)

37. The rAAV production system according to claim 35 or 36 wherein the vector genome comprises nucleic acid sequence of SEQ ID NO: 1.

38. A method of treating idiopathic dilated cardiomyopathy (DCM) in a subject in a need thereof, said method comprising administering to the subject a suspension of a rAAV of claim 1 in a formulation buffer.

39. The method according to claim 38, wherein the idiopathic DCM is an early onset idiopathic DCM.

40. The method according to claim 38, wherein the idiopathic DCM is an adult-onset form of idiopathic DCM with conduction defects.

41. A method of treating a disease associated with a dysfunctional Lamin A (LMNA) gene in a subject, said method comprising administering to the subject a suspension of a rAAV of claim 1 in a formulation buffer.

42-46. (canceled)
